(19) United States
(12) Patent Application Publication
(10) Pub. No.: US 2026/0102206 A1
(43) Pub. Date: Apr. 16, 2026

(54) CLOSED LOOP CONTROL FOR INTEROPERABLE UROLOGY OPERATING ROOM

(71) Applicant: Boston Scientific Scimed, Inc., maple grove, MN (US)

(72) Inventors: Jeffrey A. Meganck, N Grafton, MA (US); Niraj Prasad Rauniyar, Plymouth, MN (US); Hernan Altman, Tivon (IL); Christopher Stankus, Maple Grove, MN (US)

(73) Assignee: Boston Scientific Scimed, Inc., maple grove, MN (US)

(21) Appl. No.: 19/357,738

(22) Filed: Oct. 14, 2025

Related U.S. Application Data
(60) Provisional application No. 63/707,003, filed on Oct. 14, 2024.

(51) Int. Cl. A61B 18/26; A61B 1/00; A61B 1/307; A61B 17/00; A61B 18/00; G16H 40/67

(52) U.S. Cl. CPC A61B 18/26; A61B 1/00009; A61B 1/0005; A61B 1/307; G16H 40/67; A61B 2017/00199; A61B 2018/00511; A61B 2018/00642; A61B 2018/00982

(57) ABSTRACT

The disclosure provides a method for closed loop control in a urological operating room comprising: receiving, at a single control device, real-time information from a plurality of therapy consoles provisioned in an operating theater; determining whether the received information is outside a predetermined threshold range; automatically generating control signals based on a determination that the received information is outside the predetermined threshold range; sending the control signals to at least one therapy console of the plurality of therapy consoles to adjust an operational parameter of the at least one therapy console and mitigate a deviation from the predetermined threshold range; receiving real-time feedback information from the plurality of therapy consoles; iteratively updating the control signals based on the real-time feedback information; and sending the updated control signals to automatically adjust an operational parameter of the at least one therapy console and mitigate a remaining deviation from the predetermined threshold range.

### CROSS REFERENCE TO RELATED APPLICATIONS

This application claims the benefit of priority under 35 U.S.C. § 119 to U.S. Provisional Ser. No. 63/707,003 , filed Oct. 14, 2024, each of which is herein incorporated by reference in its entirety.

### TECHNICAL FIELD

The disclosure generally relates to urology operating rooms and particularly to closed loop control for an interoperable urology operating room.

### BACKGROUND

A modern urological operating room (OR) includes several complex devices each employing different technologies and systems. These several different devices are used together to perform urological procedures on a patient. The ability to leverage all these devices increases as the complexity and number of devices used to perform urological procedures increases. This ever-increasing complexity results in an increase in both the cognitive and physical burdens on the physicians, nurses, and assistants carrying out the procedure. For example, each piece of equipment used in the OR needs to be configured prior to the procedure and then monitored and controlled during the procedure. Adding to this burden, each piece of equipment typically has its own custom computing hardware configuration and display.

The various technologies with which each device in the OR relies on as well as the various computing hardware specification and display results in the need to monitor and control each device individually. As the number of devices provisioned in a urological OR increases, it becomes an untenable burden for the configure, monitor, and control each of them for the modern physician.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The disclosure provides components for an interoperable urology operating room (OR) including a centralized controller configured to interact with each device in the OR to provide for configuration, monitoring, and control (e.g., closed loop control) of the devices in a composite group or groups.

In some embodiments, the disclosure can be implemented as a method for closed loop control in a urological operating room, comprising: receiving, at a single control device, real-time information from a plurality of therapy consoles provisioned in an operating theater, each of the plurality of therapy consoles comprising processing circuitry configured for use in performing a urological procedure; determining whether the received information is outside a predetermined threshold range, wherein the received information comprises physiological information, captured scene information, radiological image information, procedural state information, or any combination thereof; automatically generating control signals based on a determination that the received information is outside the predetermined threshold range; sending the control signals from the single control device to at least one therapy console of the plurality of therapy consoles to adjust an operational parameter of the at least one therapy console and mitigate a deviation from the predetermined threshold range; receiving real-time feedback information from the plurality of therapy consoles responsive to the adjustment of the operational parameter; iteratively updating the control signals based on the real-time feedback information; and sending the updated control signals to automatically adjust an operational parameter of the at least one therapy console and mitigate a remaining deviation from the predetermined threshold range.

With further embodiments, the method can comprise wherein determining whether the received information is outside a predetermined threshold range comprises determining whether physiological information is outside a physiological threshold range, and wherein the physiological information comprises intraluminal pressure, intraluminal temperature, fluid flow rates, laser energy parameters, or any combination thereof.

With further embodiments, the method can comprise wherein determining whether the received information is outside a predetermined threshold range comprises determining whether captured scene information is outside a visual threshold range, and wherein the captured scene information comprises turbidity, clarity, image saturation, laser aiming beam visibility, or any combination thereof.

With further embodiments, the method can comprise wherein determining whether the received information is outside a predetermined threshold range comprises determining whether procedural state information is outside a procedural threshold range, and wherein the procedural state information comprises equipment activation status, time elapsed in procedure, treatment efficacy indicators, or any combination thereof.

With further embodiments, the method can comprise wherein automatically adjusting an operational parameter comprises adjusting one or more operational parameters of multiple ones of the plurality of therapy consoles in a coordinated manner to optimize performance across the multiple therapy consoles.

With further embodiments, the method can comprise wherein the plurality of therapy consoles comprises at least a fluidics unit, a laser energy console, and an endoscope, and wherein automatically adjusting an operational parameter comprises coordinating operation between at least two of the fluidics unit, laser energy console, and endoscope based on the received information.

With further embodiments, the method can comprise: determining additional information derived from the received information; and adjusting the predetermined threshold range based on the additional information to provide dynamic threshold management during the urological procedure.

With further embodiments, the method can comprise wherein automatically adjusting an operational parameter comprises adjusting fluid flow rates, laser energy parameters, scope deflection, fiber positioning, or any combination thereof.

With further embodiments, the method can comprise: monitoring changes in the received real-time feedback information over time; predicting future values of the received information based on the monitored changes; and proactively adjusting the updated control signals based on the predicted future values to prevent the received information from exceeding the predetermined threshold range.

With further embodiments, the method can comprise: determining stone composition and texture from the received information; and wherein automatically adjusting an operational parameter comprises adjusting laser energy parameters based on the determined stone composition and texture to optimize stone fragmentation while maintaining the received information within the predetermined threshold range.

With further embodiments, the method can comprise wherein the single control device is configured to automatically coordinate adjustments across multiple therapy consoles to maintain optimal procedural conditions based on the received information and real-time feedback information.

With further embodiments, the method can comprise: determining distance between a treatment device and target tissue from the received information; and wherein automatically adjusting an operational parameter comprises adjusting laser energy parameters and scope positioning based on the determined distance to maintain optimal treatment conditions.

With further embodiments, the method can comprise wherein the predetermined threshold range is dynamically adjusted during the urological procedure based on real-time analysis of the received information and real-time feedback information.

With further embodiments, the method can comprise wherein the updated control signals comprise coordinated commands that cause synchronized automatic adjustments across multiple therapy consoles to mitigate remaining deviations from the predetermined threshold range.

With further embodiments, the method can comprise: generating a composite display comprising visual indications of the received information, the automatic adjustments, and the iterative closed loop control status; and sending display control signals to cause an operating theater display to display the composite display showing real-time closed loop control operations.

In some embodiments a urological operating room closed loop control system is provided. The urological operating room closed loop control system comprising: an operating theater; a plurality of therapy consoles provisioned in the operating theater, each comprising processing circuitry configured for use in performing a urological procedure; a single control device comprising: a processor configured to: receive real-time information from the plurality of therapy consoles; determine whether the received information is outside a predetermined threshold range, wherein the received information comprises physiological information, captured scene information, radiological image information, procedural state information, or any combination thereof; automatically generate control signals based on a determination that the received information is outside the predetermined threshold range; send the control signals to at least one therapy console of the plurality of therapy consoles to adjust an operational parameter and mitigate a deviation from the predetermined threshold range; receive real-time feedback information from the plurality of therapy consoles responsive to the adjustment; iteratively update the control signals based on the real-time feedback information; and send updated control signals to automatically adjust operational parameters and mitigate remaining deviations from the predetermined threshold range.

With further embodiments, the urological operating room closed loop control system can comprise wherein the processor is further configured to automatically adjust multiple operational parameters simultaneously across different ones of the plurality of therapy consoles based on the received information and real-time feedback information to maintain optimal procedural conditions.

In some embodiments at least one machine readable storage device is provided. The at least one machine readable storage device comprising a plurality of instructions that in response to being executed by a processor of a single control device cause the single control device to: receive real-time information from a plurality of therapy consoles provisioned in an operating theater, each configured for use in performing a urological procedure; determine whether the received information is outside a predetermined threshold range, wherein the received information comprises physiological information, captured scene information, radiological image information, procedural state information, or any combination thereof; automatically generate control signals based on a determination that the received information is outside the predetermined threshold range; send the control signals to at least one therapy console of the plurality of therapy consoles to adjust an operational parameter and mitigate a deviation from the predetermined threshold range; receive real-time feedback information from the plurality of therapy consoles responsive to the adjustment; iteratively update the control signals based on the real-time feedback information; and send updated control signals to automatically adjust operational parameters and mitigate remaining deviations from the predetermined threshold range.

With further embodiments, the at least one machine readable storage device can comprise wherein the instructions when executed by the processor, further cause the single control device to: monitor changes in the real-time feedback information over time; predict future values of the received information based on the monitored changes; and proactively adjust the updated control signals based on the predicted future values to prevent the received information from exceeding the predetermined threshold range.

With further embodiments, the at least one machine readable storage device can comprise, wherein the instructions when executed by the processor, further cause the single control device to automatically coordinate adjustments across multiple therapy consoles to maintain optimal procedural conditions based on the received information and feedback signals.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A illustrates a first operating room (OR) environment in accordance with at least one embodiment.

FIG. 1B illustrates a centralized operating theater controller of the first OR environment of FIG. 1A in greater detail.

FIG. 2A, FIG. 2B, FIG. 2C, FIG. 2D, FIG. 2E, and FIG. 2F illustrate a second OR environment, provisioned for a urological procedure, in accordance with at least one embodiment.

FIG. 3A illustrates a third OR environment in accordance with at least one embodiment.

FIG. 3B illustrates a centralized operating theater controller of the third OR environment of FIG. 3A in greater detail.

FIG. 4A illustrates a first logic flow in accordance with at least one embodiment.

FIG. 4B illustrates a second logic flow in accordance with at least one embodiment.

FIG. 5 illustrates a centralized operating theater controller in accordance with at least one embodiment.

FIG. 6 illustrates a third logic flow in accordance with at least one embodiment.

FIG. 7 illustrates a fourth logic flow in accordance with at least one embodiment.

FIG. 8 illustrates a fifth logic flow in accordance with at least one embodiment.

FIG. 9 illustrates a sixth logic flow in accordance with at least one embodiment.

FIG. 10 illustrates a seventh logic flow in accordance with at least one embodiment.

FIG. 11 illustrates a computer-readable storage medium in accordance with at least one embodiment.

### DETAILED DESCRIPTION

The present disclosure is described with reference to medical devices, methods, and systems. Often, the disclosure is described with reference to surgical urological equipment and procedures. For example, in some procedures, a medical device (e.g., an endoscope, a laser fiber, a snare, a basket, etc.) may be advanced through a path or passage in a body (e.g., a ureter) to aid in removal of target tissue (e.g., a stone, or the like) from a cavity in the body (e.g., a calyx of a kidney). In another example, a medical device (e.g., an endoscope, a laser fiber, a morcellator, etc.) may be advanced through a path or passage in a body (e.g., a ureter) to aid in treatment and/or removal of target tissue (e.g., cancerous prostate tissue, or the like).

It is to be appreciated that references to a particular type of procedure, medical device, target tissue, or body passage or cavity are provided for convenience and clarity of describing the invention and are not intended to limit the claims beyond what is specified in each claim.

The terms “proximal” and “distal” may be utilized along with terms such as “parallel,” “transverse,” and “longitudinal” to describe the relative relationship and position of elements described herein. Proximal refers to a position closer to the exterior of the body (or closer to a user), whereas distal refers to a position closer to the interior of the body (or further away from the user). Further, the term “elongated” as used herein refers to an object that is substantially longer in one direction (e.g., referred to as the longitudinal direction) in relation to a perpendicular direction. For example, an object having a longer width than length could be referred to herein as elongated.

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its structure and method, together with further objects and advantages will be better understood from the following description when read in conjunction with the accompanying figures.

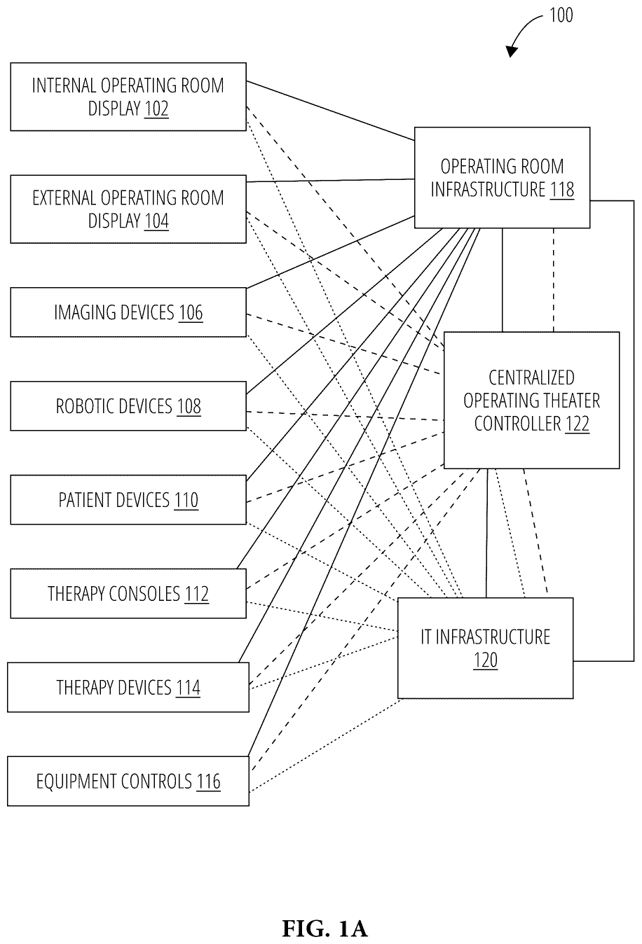

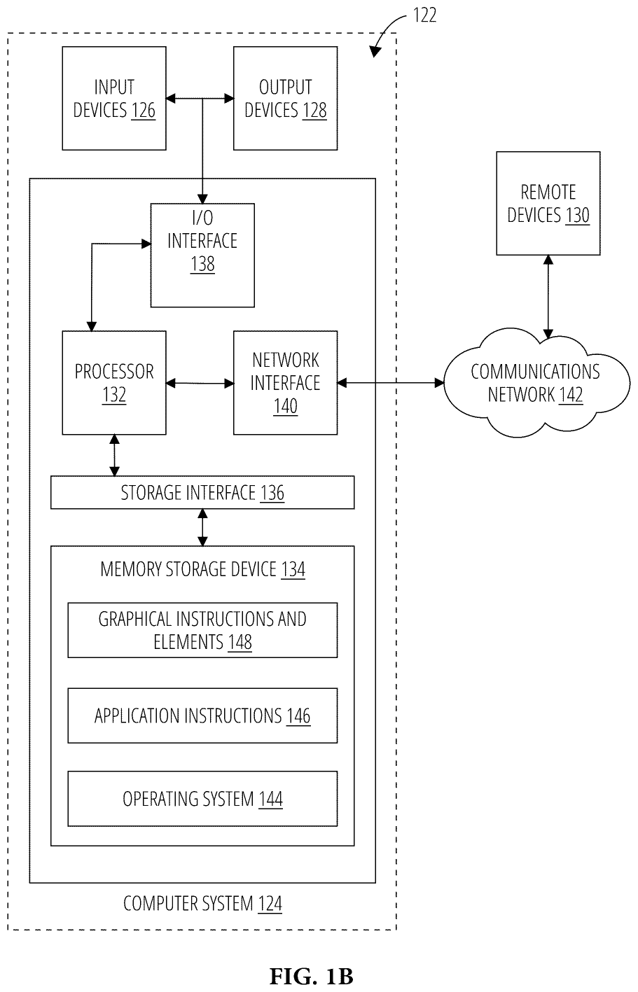

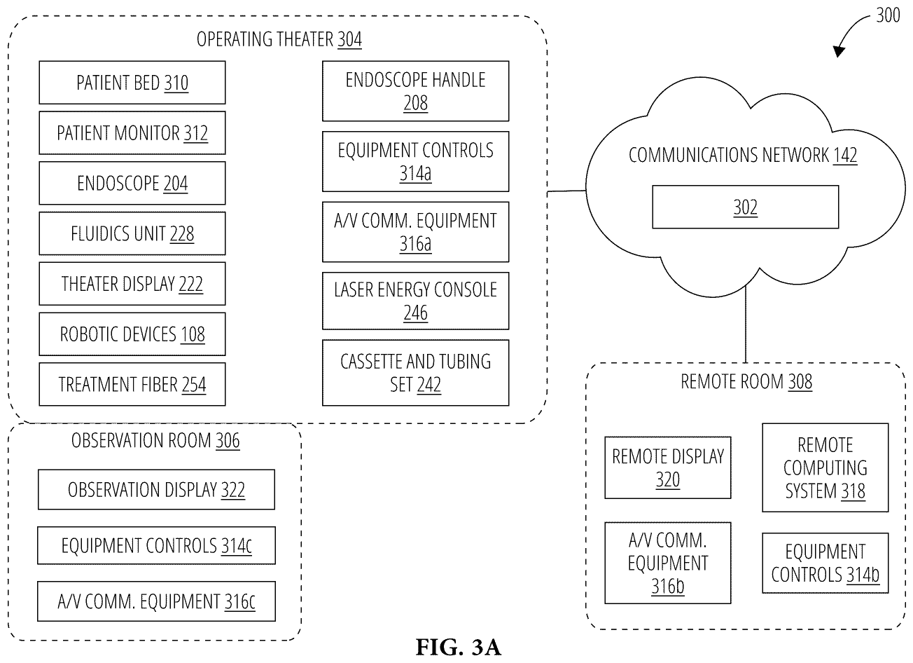

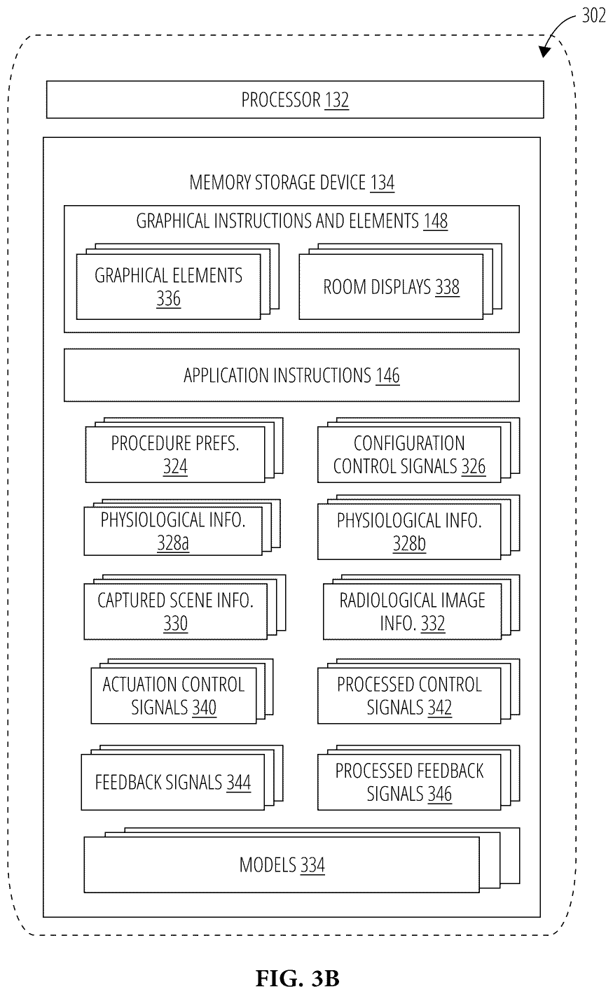

Numerous aspects of the present disclosure are now described with reference to an illustrative operating room (OR) environment 100 as depicted in FIG. 1A. The OR environment 100 will often be focused on an operating theater (or surgical suite), which is a designated room or space where the patient and procedure take place. However, the OR environment 100, and particularly, the equipment of OR environment 100 can be disposed and/or used in multiple locations including the operating theater (e.g., see FIG. 3A). Locations outside or other than the operating theater can include a viewing room, a control room, a computing infrastructure room, a proctor viewing room, a remote physician operating room, or the like). As will be appreciated, not all locations in which equipment of OR environment 100 will be deployed are sterile. Further, even in the operating theater itself there is often a delineation between a sterile and non-sterile field.

OR environment 100 can include internal and external displays. For example, OR environment 100 can include an internal operating room display 102 (or displays) arranged to display information within the operating theater. Similarly, OR environment 100 can include external operating room display 104 (or displays) arranged to display information outside the operating theater, for example, to observers, to remote physicians, to proctors, to nurses or admins outside the sterile field or in another room. Further, OR environment 100 can include imaging devices 106, robotic devices 108, patient devices 110, therapy consoles 112, therapy devices 114, equipment controls 116, operating room infrastructure 118, information technology (IT) infrastructure 120, and centralized operating theater controller 122.

With some embodiments, internal operating room display 102 can include an overhead operating theater display. As a specific example, internal operating room display 102 can include a display physically located inside of the operating theater (e.g., a wall mounted monitor, a ceiling mounted monitor, a monitor mounted on an articulating arm, or the like) that is visible to multiple observers. In some embodiments, internal operating room display 102 can include a wearable display, such as, a heads-up display, a virtual reality display, an augmented reality display, a tablet or small form factor display and associated harness to wear the display. With some embodiments, multiple wearable displays can be provided. For example, the physician can wear a head-mounted display while an assistant wears a tablet computer. In some embodiments, internal operating room display 102 can include an on-patient display, for example, a display or monitor physically attached to the patient, a display projected onto the patient, or the like.

In some embodiments, external operating room display 104 can include a monitor or monitors located outside the operating theater and configured to display information from inside the operating theater to persons outside the operating theater. For example, external operating room display 104 can include a monitor or monitors configured to display a view (or views) of the surgical suite. In some examples, the external operating room display 104 can be located proximate to (e.g., in the next room, or the like) the surgical suite while in other examples, the external operating room display 104 is in another area of the premises, off premises (e.g., in another geographical location, or the like). In some embodiments the external operating room display 104 can be configured for direct real-time viewing, recorded viewing, or both. With some embodiments, external operating room display 104 can include a monitor or monitors configured to display information from the surgical suite or components of the OR environment 100. For example, external operating room display 104 could be a monitor configured to mirror the displayed contents on a monitor associated with another piece of equipment in the surgical suite (e.g., vital monitoring equipment, therapy console, or the like. It is noted that equipment provisioned in the OR environment 100 can have its own display. Examples of these displays are described herein and can be classified as internal operating room display 102 or external operating room display 104 depending upon the location of the equipment.

Imaging devices 106 can include any of a variety of imaging devices configured to capture images of the patient, either pre-procedure, intra-procedure, or post-procedure. The imaging devices 106 can utilize any of several imaging modalities (e.g., radiography, ultrasound, tomographic, direct visualization, or the like). With some embodiments, imaging devices 106 can include a planar X-ray device, a fluoroscopy device, or the like. In some embodiments, imaging devices 106 can include an ultrasound imaging device. In some embodiments, imaging devices 106 can include a magnetic resonance imaging (MRI) device, a computed tomography (CT) scanning device, a positron emission tomography (PET)-MRI device, single-photon emission (SPE)-CT scanning device, or the like.

Robotic devices 108 can include any of a variety of robotic equipment configured to automatically or under control of a user, observe and/or assist in the procedure. For example, the robotic devices 108 can be equipment configured to provide a surgical navigation system (e.g., device, motion, body part tracking, or the like) and computing resources (e.g., processing circuitry, memory, etc.) configured to provide real-time tracking (e.g., needle tracking, therapy device tracking, or the like) or something in the operating theater (e.g., a body part, a medical device, or the like). In some examples, robotic devices 108 can include motion control, articulation, grasping to facilitate automatic movement, analysis, or control of equipment in the OR environment 100. Additionally, robotic devices can be configured to manipulate ones of the devices (e.g., therapy devices 114, or the like) automatically and/or under remote or non-contact control from a physician.

Patient devices 110 can include any equipment in direct contact with the patient, for example, anesthesia equipment, vital monitoring equipment, the surgical bed, and surgical bed accessories. With some embodiments, patient devices 110 can include equipment to provide general or local anesthesia to the patient, such as, a continuous-flow anesthetic machine, or the like. As another example, patient devices 110 can include continuous bedside monitors (e.g., for temperature, pulse, etc.), hemodynamic monitors, respiratory monitors, neurological monitors, cardiac monitors, or the like.

Therapy consoles 112 and therapy devices 114 can comprise any equipment (e.g., capital equipment, single use devices, reusable devices, or the like) arranged to perform or provide the treatment associated with the procedure. In general, therapy consoles 112 can include any capital equipment and/or infrastructure used for delivery of the desired treatment or therapy. For example, therapy consoles 112 can include visualization equipment, such as, endoscope viewing and/or control consoles. As another example, therapy consoles 112 can include fluidic consoles to provide fluid inflow and/or outflow, suction, or the like. In another example, therapy consoles 112 can include lithotripsy equipment, such as, laser consoles configured to generate laser energy to ablate, fragment, dust, or otherwise treat calculi. With another example, therapy consoles 112 can include soft tissue therapy equipment, such as, morcellation consoles, ablation consoles, resection consoles, biopsy consoles, cauterization consoles (e.g., laser, electrocautery, radio frequency (RF) cautery, or the like).

Therapy devices 114 can include any device used with the therapy consoles 112 to affect the treatment or procedure. For example, therapy devices 114 can include endoscopes, such as, a ureteroscope, a cystoscope, a nephroscope, or a resectoscope. The endoscopes can be electronic or optical and can be configured to visualize the anatomy in minimally invasive procedures. With some examples, therapy devices 114 can include retrieval devices (e.g., baskets, snares, loops, hooks, pinchers, or the like). In some examples, therapy devices 114 can include optical fibers to convey laser energy to a treatment site, morcellation devices, energy delivery devices (e.g., thermal, electric, RF, or the like). In some examples, therapy devices 114 can include post-procedure or healing devices, such as, stents and stent delivery devices, or the like. Any of the therapy devices 114 can be single use devices, reusable devices, or therapy devices 114 can include a combination of single use and reusable devices.

Equipment controls 116 includes all equipment and/or interfaces used to control equipment in OR environment 100 and/or facilitate exchange of data between equipment in OR environment 100. Examples of such controls are provided throughout this disclosure.

Operating room infrastructure 118 can include any equipment built into the physical infrastructure of the surgical suite. For example, operating room infrastructure 118 can include operating theater lighting (e.g., wall mounted, ceiling mounted, mounted to an articulating arm, or the like) configured to provide illumination of the room and/or surgical field. In some examples, operating room infrastructure 118 can include image and/or audio capture devices (e.g., video cameras, or the like). In some examples, operating room infrastructure 118 can include centralized air, water, and/or gas supply lines (e.g., suction, filtered water, oxygen, etc.) In some examples, operating room infrastructure 118 can include central waste collection systems (e.g., floor drain, or the like). In some examples, operating room infrastructure 118 can include warming systems (e.g., warming oven, or the like) configured to warm consumables used during a procedure. With some examples, operating room infrastructure 118 can include electrical power supplies and can include hardwired or mobile power supplies.

IT infrastructure 120 can include any structure and equipment used for the transmission, storage, and/or processing of data used in the procedure. For example, IT infrastructure 120 can include servers, data storage arrays, data centers, wire and/or wireless communication cabling and equipment, medical record data storage devices, or the like.

As depicted, the components of OR environment 100 are coupled to centralized operating room infrastructure 118 and IT infrastructure 120. For example, internal operating room display 102, external operating room display 104, imaging devices 106, robotic devices 108, patient devices 110, therapy consoles 112, and/or therapy devices 114 can be configured to receive and/or transmit data (e.g., information elements, control signals, etc.) via IT infrastructure 120. Such exchange of data can be unidirectional or bidirectional. Examples of this are provided throughout the disclosure.

Further, components of OR environment 100 that contain electrical and/or electromechanical elements can be coupled to a source of power via operating room infrastructure 118. Similarly, components of the OR environment 100 may be coupled to fluid and/or gas supplies via operating room infrastructure 118. For example, internal operating room display 102, external operating room display 104, imaging devices 106, robotic devices 108, patient devices 110, therapy consoles 112, and/or therapy devices 114 can be configured to receive electrical power, gas supply, water inflow and/or outflow supply, vacuum supply, or the like in any combination via operating room infrastructure 118.

Lastly, centralized operating theater controller 122 can be coupled to any equipment or components of the OR environment 100 via IT infrastructure 120. FIG. 1B illustrates an example centralized operating theater controller 122. In general, the centralized operating theater controller 122 can be configured to send and/or receive data to and/or from equipment (e.g., therapy consoles internal operating room display 102, external operating room display 104, imaging devices 106, robotic devices 108, patient devices 110, therapy consoles 112, therapy devices 114, equipment controls 116, and/or operating room infrastructure 118). Further, centralized operating theater controller 122 can be configured to process data, infer information from data, and execute instructions to implement methods described herein.

It is noted that centralized operating theater controller 122 is depicted in FIG. 1A and FIG. 1B as a single component. However, in practice centralized operating theater controller 122 can be multiple components or a single component. Further, centralized operating theater controller 122 can be embodied (e.g., housed) in one of the other pieces of equipment depicted in FIG. 1A. For example, one of the therapy consoles 112 can include hardware as depicted in FIG. 1B and can be configured as outlined herein to operate as centralized operating theater controller 122.

As depicted in FIG. 1B, centralized operating theater controller 122 can include computer system 124, input devices 126, output devices 128, and/or remote devices 130.

The computer system 124 may include processor 132 and a memory storage device 134 coupled to the processor 132 via a storage interface 136. In general, processor 132 can be processing circuitry configured to execute instructions stored on memory storage device 134. For example, processor 132 can be a central processing unit (CPU), a graphics processing unit, a machine learning (ML) processing unit, or a combination of these. The processor 132 may include specialized processing units such as integrated system (bus) controllers, memory management control units, floating point units, graphics processing units, neural processing units, digital signal processing units, etc. Processor 132 can be an off the shelf CPU or can be custom designed processing circuitry (e.g., an application specific integrated circuit (ASIC), or the like).

Memory storage device 134 may include computer-readable storage media or devices configured to store data. Such data can take a variety of forms or data structures. One form of such data is machine code (also referred to as “instructions”) that is executable by processor 132. In some examples, memory storage device 134 can be physical memory on which information or data readable by a processor (e.g., processor 132) may be stored. Thus, a computer-readable storage medium may store instructions for execution by one or more processors (e.g., processor 132, or the like) including instructions for causing the processor(s) to perform steps or stages consistent with the embodiments described herein.

The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media. Although depicted in FIG. 1B, memory storage device 134 need not be collocated with processor 132 and can for example, be accessible via a network.

In some embodiments, the storage interface 136 may be configured to connect to memory storage device 134 via memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Processor 132 can be disposed in communication with input devices 126 and output devices 128 via I/O interface 138. The I/O interface 138 may employ communication protocols and/or methods such as, without limitation, audio, analog, digital, stereo, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), Ethernet, Bluetooth, cellular, etc. Using the I/O interface 138, computer system 124 may communicate with input devices 126 and output devices 128. In general, input devices 126 can be any control or input device used to provide input to equipment in the OR environment 100.

In some embodiments, input devices 126 can include devices configured to receive input from a user of the OR environment 100 (e.g., a physician, a nurse, an assistant, a technician, or the like). For example, input devices 126 can include wearable controls such as watches, armbands, headphones, footwear, or the like. In another example, input devices 126 can include touch-based controls such as foot pedals, switches, triggers, touch screens, buttons, or the like. In some examples, input devices 126 can include non-touch-based controls such as voice activation controls, gesture-based controls, eye movement-based controls, or the like. These various input devices implemented as input devices 126 can be equipment controls 116 described above or can be inputs to other computing equipment (e.g., imaging devices 106, robotic devices 108, patient devices 110, therapy consoles 112, therapy devices 114, etc.)

With some embodiments, output devices 128 can include internal operating room display 102 and/or external operating room display 104. With some embodiments, output devices 128 can include non-display outputs such as, audio output, flashing light output, haptic output, or the like. Further, in some examples, ones of input devices 126 and/or output devices 128 can be combined. For example, a touch screen display can be configured as both input devices 126 and output devices 128.

It is to be appreciated that although input devices 126 and output devices 128 are depicted as included (or packaged) with computer system centralized operating theater controller 122, they may not be explicitly part of centralized operating theater controller 122 but could be I/O devices of another computer system described herein with which centralized operating theater controller 122 is configured to communicate.

As noted, memory storage device 134 may store instructions executable by processor 132. This can include various types of instructions. For example, memory storage device 134 can store an operating system 144 and/or application instructions 146. Further, memory storage device 134 can store graphical instructions and elements 148 (e.g., user interface elements, graphical information elements, etc.) In various embodiments, the operating system 144 may facilitate resource management and operation of the computer system 124 and facilitate communicative coupling with input devices 126, output devices 128, and other equipment in the OR environment 100 coupled via IT infrastructure 120. Examples of operating systems include, without limitation, UNIX®, UNIX-like system distributions (e.g., BERKELEY SOFTWARE DISTRIBUTION®(BSD), FREEBSD®, NETBSD®, OPENBSD, etc.), LINUX® DISTRIBUTIONS (e.g., RED HAT®, UBUNTU®, KUBUNTU®, etc.), IBM®OS/2®, MICROSOFT®WINDOWS®, APPLE®IOS®, GOOGLE™ ANDROID™, or the like.

The application instructions 146 may include instructions that when executed by the processor 132 cause centralized operating theater controller 122 to perform one or more techniques, steps, procedures, and/or methods described herein, such as to send and/or receive data to and/or from equipment of OR environment 100, process the data, infer information from ML models, execute algorithms based on the data, and send and/or receive control signals to and/or from the equipment in the OR environment 100.

The graphical instructions and elements 148 may include instructions that when executed by the processor 132 cause the processor 132 to facilitate rendering and display of information on displays in patient devices 110. The graphical instructions and elements 148 can also include the rendered graphical elements (or frames) to be displayed. For example, graphical instructions and elements 148 can be configured to provide cursors, icons, checkboxes, menus, scrollers, windows, widgets, etcetera. Such graphical instructions and elements 148 can provide an interface (e.g., analog, or digital) to equipment of the OR environment 100 or a display of settings, parameters, status, or other information related to the equipment.

In some embodiments, the processor 132 may be in communication with remote devices 130 via network interface 140 and communications network 142. Remote devices 130 can be any of a variety of computing devices (e.g., cloud computing resources, cloud storage resources, remote servers, remote sensors, remote workstations, etc.) The network interface 140 may permit communication between computer system 124 and remote devices 130 via the communications network 142. To that end, the network interface 140 may employ connection protocols including, without limitation, direct connect, Ethernet, Transmission Control Protocol/Internet Protocol (TCP/IP), token ring, Wi-Fi, etc. The communications network 142 can be implemented as one of the different types of networks, such as intranet or Local Area Network (LAN), Closed Area Network (CAN), the Internet, and such. The communications network 142 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 142 may include a variety of network devices, including routers, bridges, servers, computing devices, storage devices, etcetera.

Many pieces of equipment in OR environment 100 can include computing components or a computing system. For example, therapy consoles 112 often include computing systems including computing components like described above with respect to centralized operating theater controller 122 and particularly computer system 124. Accordingly, computing components of such devices are often discussed with reference to the components of computer system 124. However, this is done for convenience only. It is to be appreciated that these other computer systems may include some or all the components of computer system 124 and may include different components or additional components that are not shown in FIG. 1B. In general, however, such computer system will include a processor (e.g., like processor 132) and a memory storage device (e.g., like memory storage device 134) that includes instructions.

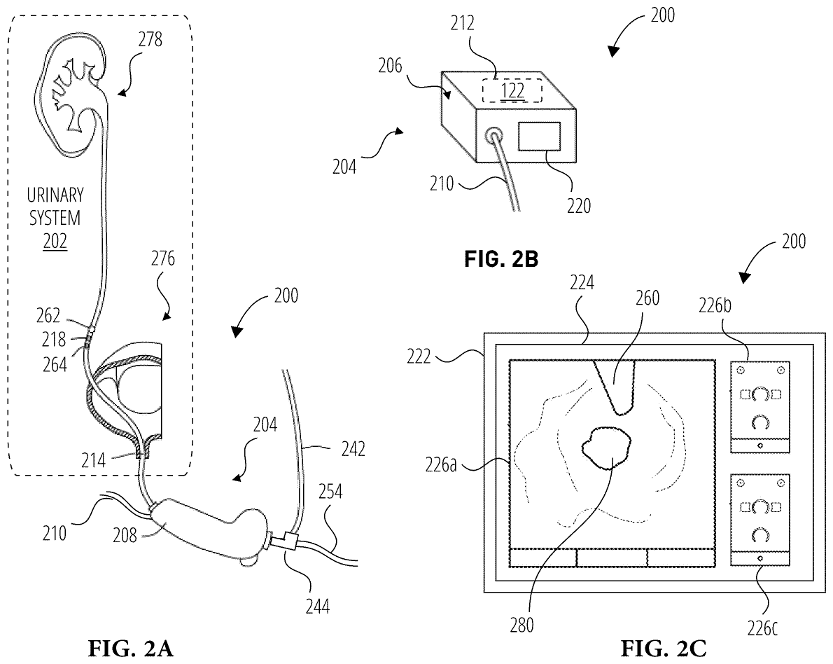

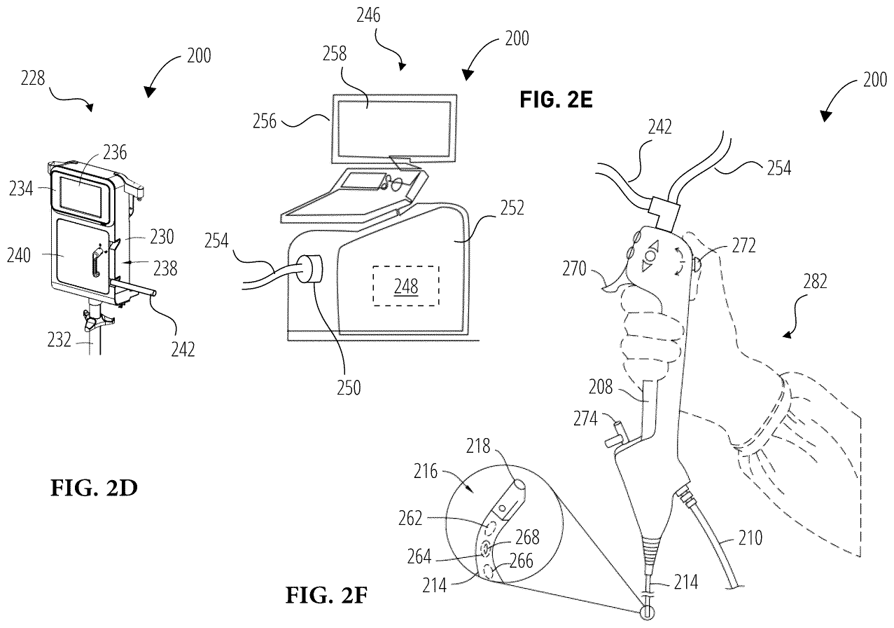

As noted above, OR environment 100 can be provisioned and implemented to perform urological procedures. For example, FIG. 2A to FIG. 2E illustrates an OR environment 200, which can be implemented using components from OR environment 100 described above to perform a urological procedure. OR environment 200 is described with respect to a lithotripsy procedure to treat urinary calculi (referred to as “stone”) in a urinary system 202. However, this is not intended to be limiting and OR environment 100 and/or OR environment 200 could be implemented to perform other urological procedures, such as, for example, percutaneous nephrolithotomy (PCNL), benign prostatic hyperplasia (BPH), transurethral resection of the prostate (TURP), etc.

OR environment 200 can be implemented with an endoscope 204, such as, a ureteroscope. Endoscope 204 can include an endoscope console 206 (e.g., one of therapy consoles 112), which can be configured to operate with an endoscope handle 208 (e.g., one of therapy devices 114). The endoscope console 206 and endoscope handle 208 can be coupled via connection cable 210. FIG. 2A, and FIG. 2F illustrate endoscope handle 208 in OR environment 200 while FIG. 2B illustrates endoscope console 206. Endoscope 204 can be coupled to a source of power via operating room infrastructure 118. Further, the connection cable 210 can be configured to provide power from endoscope console 206 to endoscope handle 208 and to provide exchange of data between endoscope console 206 and endoscope handle 208.

Endoscope console 206 can include computing system 212 (e.g., like computer system 124) which itself can include (or be coupled to) a display 220 (e.g., touch screen display, or the like). Endoscope console 206 can also include input and/or output devices (not shown), such as buttons, lights, switches, etc. Further, OR environment 200 can include computing components configured to operate as the centralized operating theater controller 122 described above with respect to FIG. 1A and FIG. 1B. With some embodiments, as shown, centralized operating theater controller 122 can be integrated into one of the therapy consoles 112. Accordingly, endoscope console 206 can include computing system 212 and computer system 124.

In some embodiments a single computing system (e.g., computer system 124, or the like) can be provided as part of endoscope console 206 and configured to operate as both computing system 212 and centralized operating theater controller 122. For example, FIG. 2B illustrates centralized operating theater controller 122 into integrated endoscope console 206 as part of computing system 212. However, this is not intended to be limiting and centralized operating theater controller 122 could be a separate computing system disposed in the housing of endoscope console 206 or could be integrated into another therapy consoles 112 provisioned in the OR environment 200 (e.g., theater display 222, fluidics unit 228, laser energy console 246, or the like). Or as depicted in OR environment 100, centralized operating theater controller 122 of OR environment 200 could be a stand-alone component provisioned in OR environment 200. In some embodiments, centralized operating theater controller 122 can be a cloud computing system (e.g., computing as a service (CaaS), or the like) accessible via communications network 142. In such an example, equipment in OR environment 200 (e.g., computing system 212 of endoscope console 206, or the like) can include network interfaces (e.g., network interface 140) to permit communication with centralized operating theater controller 122 on communications network 142.

Operation of centralized operating theater controller 122 in OR environment 200 is described in greater detail below. However, in general, centralized operating theater controller 122 is configured to provide data communication between devices provisioned in OR environment 200. For example, centralized operating theater controller 122 can be configured to provide data communication between endoscope 204, display 220, fluidics unit 228, laser energy console 246, and their associated therapy devices 114, or any combination of these components. Further, centralized operating theater controller 122 can be configured to process such data as outlined herein (e.g., send and receive control signals between devices based on (or responsive to) the communicated data, execute algorithms on the communicated data as part of controlling operation of the components, or the like).

Endoscope 204 can include an elongated shaft 214 coupled to endoscope handle 208, which can be used to access a patient's bladder 276 and/or kidney 278. In such a procedure, the endoscope 204, and particularly, a distal end 216 of the elongated shaft 214 is inserted into the bladder 276 via the urethra and can be further inserted into the kidney 278 via the ureter, where it can be used to diagnose and/or treat a variety of problems in the urinary system 202. The endoscope 204 can include a camera 218 disposed on the distal end 216 of the elongated shaft 214. The camera 218 can be used to provide a visual feed on a display screen. For example, images captured by camera 218 can be rendered and displayed on display 220 of endoscope console 206. Additionally, OR environment 200 can be provided with several other displays (e.g., internal operating room display 102 and/or external operating room display 104) that can be configured to display images and/or video captured by camera 218 of endoscope 204. For example, centralized operating theater controller 122 can be configured, or rather, can include application instructions 146 stored in memory storage device 134 that when executed by processor 132 cause centralized operating theater controller 122 to receive data comprising indications of image frames captured by camera 218, process the image frames, and send the processed image frames to theater display 222 for display. Such communication can be facilitated by communicative connection between the equipment in OR environment 200 via IT infrastructure 120.

For example, FIG. 2C illustrates a theater display 222 (or operating theater display), in which is depicted a composite display 224 having a grouping of individual graphical elements 226a to 226c. For example, theater display 222 shows composite display 224 having graphical element 226a, 226b, and 226c where graphical element 226a depicts a view of an image captured by camera 218. It is to be appreciated that this view could be a live view or a recorded view and various examples of each are provided herein.

OR environment 200 can further include a fluidics unit 228 (e.g., as one of therapy consoles 112). Fluidics unit 228 can be coupled to endoscope 204 and called on to provide fluid flow to the distal end 216 of the elongated shaft 214. For example, fluidics unit 228 could be utilized to clear the visual field of the camera 218. Fluidics unit 228 can include a console 230. In some examples, console 230 can be mounted on pole 232 attached to a mobile base (not shown). In other examples, console 230 can be free standing, table mounted, or the like. Console 230 can include computing system 234 (e.g., like computer system 124) which itself can include a display 236 (e.g., touch screen display, or the like). Fluidics unit 228 can also include input and/or output devices (not shown), such as, buttons, lights, switches, etc.

Console 230 can include an interface (not shown) with connection sockets and/or busses to which computing system 234 can be communicatively coupled to centralized operating theater controller 122 via IT infrastructure 120. Such interface can also couple fluidics unit 228 to a source of power via operating room infrastructure 118. For example, a connection cable (not shown) could couple computing system 234 to centralized operating theater controller 122 in endoscope console 206 (e.g., via IT infrastructure 120, or the like) and couple fluidics unit 228 to power provided by operating room infrastructure 118.

Fluidics unit 228 can include a pump 238 (disposed in console 230). The pump can be configured to provide fluid flow when requested by the user (e.g., via the endoscope handle 208, or the like). Fluidics unit 228 can be configured to operate with a cassette and tubing set 242 (e.g., one of therapy devices 114, or the like). The cassette and tubing set 242 can be disposed in console 230 via door 240. Further, the cassette and tubing set 242 can be coupled to a source of fluid (not shown) and to the endoscope handle 208 (e.g., via fluid port 244 as shown in FIG. 2A, or the like). In some examples, the fluid source can be saline bags, or the like.

The computing system 234 can control pump 238 (e.g., responsive to input from endoscope 204, endoscope handle 208, responsive to sensor(s) output, responsive to control signals from centralized operating theater controller 122, or the like) to cause fluid to flow to the distal end 216 of the elongated shaft 214 via a working channel or dedicated fluid channel (not shown) in the elongated shaft 214. Additionally, in some embodiments, fluidics unit 228 can include a heater and/or a chiller to heat and/or cool the fluid supplied to the treatment site via the elongated shaft 214. Fluid flow to the treatment site (e.g., body cavity, or the like) in the urinary system 202 where the stone 280 is located affects the pressure inside the body cavity. This pressure is referred to herein as intraluminal pressure (ILP).

During an example lithotripsy procedure, blood and/or debris may be present in the body cavity, which may negatively affect image quality captured by the endoscope 204. Fluid flow (e.g., irrigation fluid flow) from fluidics unit 228 may be used to flush the body cavity to improve the image quality. Further, as laser energy (described below) can be used to fragment, ablate, dust, or otherwise treat the stone, heat may be generated at the treatment site. Fluid flow can be used to control the temperature of the treatment site to avoid damage or injury to adjacent tissue.

OR environment 200 can further include a laser energy console 246 (e.g., as one of therapy consoles 112) provisioned in OR environment 200. Continuing with the example discussed above where OR environment 200 is provisioned for a lithotripsy procedure, laser energy console 246 could be a medical laser console, such as, a Holmium (Ho) laser or a Thulium (Tm) fiber laser console. As another example, laser energy console 246 could be a tissue ablation console (e.g., electronic ablation, RF ablation, etc.). With yet another example, laser energy console 246 could be a laser morcellator. With some embodiments, OR environment 200 could be provisioned with multiple laser energy consoles 246 (e.g., a morcellator and stone dusting console, or the like). Further, although not shown, OR environment 200 could include other consoles appropriate for the procedure to be performed in OR environment 200.

Laser energy console 246 can include a laser generator 248 and an optical coupler 250, both disposed in a housing 252. The laser generator 248 can be configured to generate laser energy appropriate for treating a target tissue (e.g., stone 280). A treatment fiber 254 (e.g., one of therapy devices 114, or the like) can be coupled to the laser generator 248 via the optical coupler 250. In some embodiments, laser generator 248 can comprise multiple light sources (e.g., a treatment beam, multiple treatment beams, an aiming beam, a diagnostic beam, etc.). Further, laser generator 248 can often include various optical components and sensors configured to measure characteristics or qualities of the laser energy and its effect on the stone 280, or adjacent tissue.

Laser energy console 246 can include computing system 256 (e.g., like computer system 124) which itself can include a display 258 (e.g., touch screen display, or the like). Laser energy console 246 can also include input and/or output devices (not shown), such as, buttons, lights, switches, foot pedals, etc.

Housing 252 can include an interface (not shown) with connection sockets and/or busses to which computing system 256 can be communicatively coupled to centralized operating theater controller 122 via IT infrastructure 120. Such interface can also couple laser energy console 246 to a source of power via operating room infrastructure 118. For example, a connection cable (not shown) could couple computing system 256 to centralized operating theater controller 122 in endoscope console 206 (e.g., via IT infrastructure 120, or the like) and couple laser energy console 246 to power provided by operating room infrastructure 118.

During an example lithotripsy procedure, the treatment fiber 254 can be inserted into port 244 of endoscope handles 208 and pushed through a working channel (not shown) of elongated shaft 214 such that a distal end 260 of the treatment fiber 254 can be positioned proximate to stone 280 in urinary system 202. For example, graphical element 226a depicts an image captured by camera 218 of endoscope 204 in which the distal end 260 of the treatment fiber 254 and stone 280 are shown in the urinary system 202. Laser generator 248 can generate laser energy, which is optically coupled to treatment fiber 254 via the optical coupler 250. The laser energy is conveyed through the treatment fiber 254 and emitted from the distal end 260, where it may be incident on stone 280 to cause the stone 280 to be treated (e.g., ablated, fragmented, dusted, etc.).

The computing system 256 can control laser generator 248 (e.g., responsive to input from endoscope 204, endoscope handle 208, responsive to an input device like a foot pedal, responsive to sensor(s) output, responsive to control signals from centralized operating theater controller 122, or the like) to cause the laser generator 248 to generate laser energy having parameters appropriate for the treatment to be generated. Examples of this are described in greater detail below.

In some embodiments, the working channel in which the treatment fiber 254 is inserted is different from the working channel through which fluid supplied by fluidics unit 228 flows. With some embodiments, the working channel in which the treatment fiber 254 is inserted is the same working channel through which fluid supplied by fluidics unit 228 flows.

A user (e.g., physician, a nurse, an assistant, or the like) of OR environment 200 can configure (e.g., enter treatment therapy details, or the like) via the computing components of each respective one of therapy consoles 112 provisioned in OR environment 200. For example, a user can configure endoscope 204 via computing system 212, configure fluidics unit 228 via computing system 234, and configure laser energy console 246 via computing system 256. As another example, a user can configure individual ones of the components of OR environment 200 via centralized operating theater controller 122.

Further, a user can perform a treatment via one or more of the therapy devices 114 described above. For example, endoscope 204 includes endoscope handle 208, which is depicted in use by a user 282 in FIG. 2F. As outlined above, the endoscope handle 208 can be fluidly coupled to fluidics unit 228 via cassette and tubing set 242. Further, a treatment fiber 254 can be disposed through endoscope handle 208 and into urinary system 202. It is to be appreciated that although FIG. 2F depicts a user 282 manipulating endoscope handle 208 during a procedure in OR environment 200, other embodiments may provide robotic, non-manual, or non-touch-based control of devices, such as, endoscope handle 208. Further, it is to be appreciated that the user need not be in the operating theater to control the therapy consoles 112 (e.g., see FIG. 3A).

In some embodiments, the endoscope 204 may include one or more sensors, which can be disposed proximate the distal end 216 of the elongated shaft 214. For example, FIG. 2F depicts pressure sensor 262 at the distal end 216 of the elongated shaft 214. Pressure sensor 262 can be configured to measure an intraluminal pressure (ILP) within the treatment site (see FIG. 2A). The endoscope 204 may also include other sensors such as, for example, a temperature sensor 264, a grating 266 (e.g., a Fiber Bragg grating, or the like) to detect stresses, and/or an antenna or electromagnetic sensor 268 (e.g., a position sensor).

Further, as noted, the endoscope 204 includes at least one camera 218 disposed at the distal end 216 of the elongated shaft 214 to provide a visual feed (e.g., as shown in graphical element 226a, or the like) to the user. The endoscope handle 208 can have a fluid flow on/off switch 270, which allows the user 282 to control when fluid is flowing through the elongated shaft 214 and into the treatment site. The endoscope handle 208 may further include other buttons 272 that perform other functions (e.g., control other devices provisioned in OR environment 200, or the like). For example, in some embodiments, the endoscope handle 208 may include buttons 272 to control the temperature of the fluid. In some embodiments, the endoscope handle 208 may also include a drainage port 274, which may be connected to a drainage system (e.g., of operating room infrastructure 118) and can be configured to provide a path for return flow of fluid from the treatment site.

As indicated above, all components of the urological OR suite need not reside in the operating theater. For example, equipment and users of OR environment 100 or OR environment 200 could be in different room, buildings, sites, or geographic locations. FIG. 3A depicts an OR environment 300 having multiple rooms. In some embodiments, OR environment 300 can be implemented to provide remote proctorship and/or telesurgery. OR environment 300 provides an advantage in that highly trained specialists can be utilized to observe, supervise, train, troubleshoot, and/or otherwise facilitate procedures across OR suites without having to travel to each suite.

FIG. 3A depicts OR environment 300 with operating theater 304, observation room 306, and remote room 308. In general, operating theater 304 is the room where the patient and the bulk of the equipment used to monitor and treat the patient are located. Observation room 306 can be a room proximate too but separate from operating theater 304. For example, observation room 306 can be outside the sterile field, separated from operating theater 304 by a glass wall or window, or the like. Remote room 308 can be a room in the same building as operating theater 304 and observation room 306, in another building from operating theater 304 and observation room 306, or even in another physical or geographic location (e.g., different facility site, different city, different country, partner facility site, etc.)

Communication and interoperability between the equipment in rooms of OR environment 300 is facilitated by centralized operating theater controller 302. Centralized operating theater controller 302 can be implemented as centralized operating theater controller 302 and can include all the components, structure, and features with which centralized operating theater controller 302 is described and attributed herein. As depicted, centralized operating theater controller 302 is provided as a cloud accessible computing system (e.g., in communications network 142). However, centralized operating theater controller 302 could be provided as part of endoscope 204 like described above in FIG. 2B, or any other piece of equipment in OR environment 300.

Operating theater 304, observation room 306, and remote room 308 can be connected via IT infrastructure 120, which can include communications network 142. As such, centralized operating theater controller 302 can communicate with equipment in each room.

OR environment 300 is described with reference to the OR environment 200 described above for consistency and clarity. However, OR environment 300 could be provisioned with equipment other than described herein. Continuing with the example lithotripsy procedure described above, operating theater 304 can include patient bed 310, patient monitor 312, endoscope 204, fluidics unit 228, laser energy console 246, and audio-visual communication equipment 316a (A/V equipment). Operating theater 304 can also be provisioned with theater display 222, equipment controls 314a, and/or robotic devices 108. However, OR environment 300 could be implemented where 304 is not provisioned with theater display 222 and equipment controls 314a, for example, where users needing theater display 222 and equipment controls 314a are not located in operating theater 304. As another example, operating theater 304 could be provisioned with robotic devices 108 where control of equipment in operating theater 304 (e.g., endoscope 204, fluidics unit 228, laser energy console 246, or the like) from remote room 308 is implemented.

Remote room 308 can include remote computing system 318 (e.g., like computer system 124, or the like) including remote display 320 and A/V equipment 316b. Remote room 308 can further include equipment controls 314b. For example, where remote room 308 is used to control (e.g., telesurgery, or the like) equipment in operating theater 304, remote room 308 can include equipment controls 314b. Observation room 306 can include A/V equipment 316c and may also include equipment controls 314c and/or observation display 322.

Equipment controls 314a, 314b, and 314c can include any of equipment controls 116 described herein. A/V equipment 316a, 316b, and 316c can be cameras, microphones, or other equipment configured to provide a view of the procedure and/or communication between rooms. For example, A/V equipment 316a, 316b, and 316c can include a microphone and speaker (e.g., fixed in place in the respective room, wearable, etc.) to permit audio communication between rooms. A/V equipment 316a can include a camera positioned to provide a view of the patient and endoscope handle 208, treatment fiber 254, cassette and tubing set 242, and/or other therapy devices 114. With some embodiments, A/V equipment 316b and 316c can include a camera arranged to provide views of the occupants of each respective room.

Further, various therapy devices 114 can be provided in operating theater 304, for example, endoscope handle 208, cassette and tubing set 242, and treatment fiber 254 can be provided in operating theater 304.

During operation, centralized operating theater controller 302 can provide for monitoring, parameter adjustment, and/or control of equipment in operating theater 304 by users in operating theater 304, observation room 306, and/or remote room 308. For example, centralized operating theater controller 302 can provide monitoring of patient bed 310, patient monitor 312, endoscope 204, fluidics unit 228, and laser energy console 246 via observation display 322 in observation room 306. Further, centralized operating theater controller 302 can provide control of endoscope 204, fluidics unit 228, and laser energy console 246 via equipment controls 314b in remote room 308. For example, a specialist physician can be assigned the responsibility of controlling endoscope 204 via the endoscope handle 208, fluidics unit 228, and laser generator 248, while a nurse can be positioned in operating theater 304 and assigned the responsibility of monitoring patient bed 310 and patient monitor 312. As such, centralized operating theater controller 302 can be configured to control (e.g., robotic devices 108, or the like) equipment in operating theater 304 from inputs and/or control signals received from equipment controls 314b in remote room 308.

FIG. 3B illustrates the centralized operating theater controller 302 shown in FIG. 3A in greater detail. It is to be appreciated that this figure may not depict all elements of centralized operating theater controller 302. For example, elements such as the operating system 144, interconnects, or the like are omitted for clarity. Centralized operating theater controller 302 includes at least a processor 132 and memory storage device 134. In general, memory storage device 134 stores instructions (e.g., application instructions 146, or the like) executable by the processor 132, which when executed cause the centralized operating theater controller 302 to provide remote proctoring and/or telesurgery functionality of any OR environment described herein.

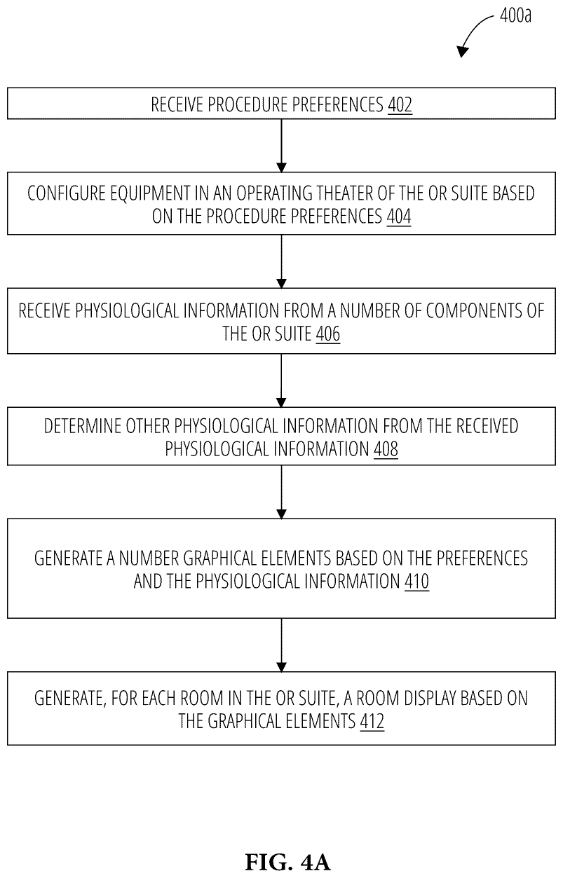

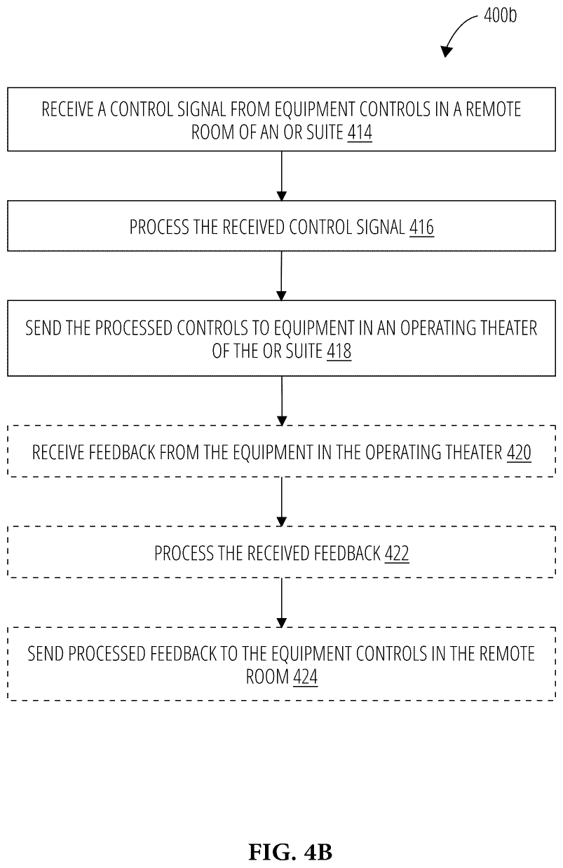

Centralized operating theater controller 302 is described with reference to FIG. 4A and FIG. 4B. FIG. 4A and FIG. 4B illustrate logic flows 400a and 400b, respectively. These logic flows can be carried out by centralized operating theater controller 302 to provide remote proctoring and/or telesurgery. In some embodiments, centralized operating theater controller 302 can carry out logic flows 400a and 400b simultaneously.

Logic flow 400a can begin at block 402. At block 402 “receive procedure preferences” preferences for the treatment procedure being carried out can be received. In some examples, these preferences can be based on the type of procedure and equipment provisioned in the OR suite. In further examples, these preferences can be based on a physician in the OR suite, established clinic preferences, or the like. Processor 132 can execute application instructions 146 to cause centralized operating theater controller 302 to receive procedure preferences 324 (e.g., from a data center, from local storage, from a cloud-based storage location, from equipment controls 314a, equipment controls 314b, or the like). For example, processor 132 can execute application instructions 146 to receive procedure preferences 324 from user (e.g., proctor, physician, etc.) in remote room 308 via equipment controls 314b.

Continuing to block 404 “configure equipment in an operating theater of the OR suite based on the procedure preferences” equipment in the operating theater of OR environment 300 can be configured based on the procedure preferences. Processor 132 can execute application instructions 146 to cause centralized operating theater controller 302 to configure or otherwise adjust settings on equipment (e.g., endoscope 204, fluidics unit 228, laser energy console 246, etc.) based on the procedure preferences 324. For example, procedure preferences 324 may be parameters for laser energy to be generated by laser energy console 246. As such, processor 132 can execute application instructions 146 to generate configuration control signals 326 and send configuration control signals 326 to laser energy console 246 to cause laser energy console 246 to be placed in a configuration wherein laser energy having the desired parameters will be generated. As another example, procedure preferences 324 may be parameters for fluid flow supplied by fluidics unit 228 and/or ILP. As such, processor 132 can execute application instructions 146 to generate configuration control signals 326 and send configuration control signals 326 to fluidics unit 228 to cause fluidics unit 228 to be placed in a configuration wherein fluid from cassette and tubing set 242 will flow according to the parameters.

Continuing to block 406 “receive physiological information from a number of components of the OR suite” physiological data can be received from equipment of the OR environment 300. Processor 132 can execute application instructions 146 to cause centralized operating theater controller 302 to receive physiological information 328a from devices and/or consoles of OR environment 300. For example, processor 132 can execute application instructions 146 to receive data (e.g., an information element, sensor output signals, or the like) comprising indications of an intensity of laser energy (e.g., diagnostic energy, aiming energy, treatment energy, or the like) generated by laser generators 248. With some embodiments, processor 132 can execute application instructions 146 to receive physiological information 328a from computing system 256 of laser energy console 246. With some embodiments, processor 132 can execute application instructions 146 to determine the intensity based on signals received from sensors (not shown) of laser energy console 246 where the sensors are configured to measure qualities and/or characteristics of the laser energy.

In some embodiments, processor 132 can execute application instructions 146 to receive physiological information 328a comprising indications of turbidity and/or clarity of scene(s) captured by camera 218 of endoscope 204. In some embodiments, processor 132 can execute application instructions 146 to receive captured scene information 330 comprising image frames of the scene(s). In some embodiments, processor 132 can execute application instructions 146 to receive physiological information 328a comprising indications of an ILP, flow rate of fluidics unit 228, or both.

In some embodiments, processor 132 can execute application instructions 146 to receive physiological information 328a comprising indications of a distance between the distal end 260 of the treatment fiber 254 and the stone 280, a composition of the stone 280, and/or a texture of the stone 280. With some embodiments, laser energy console 246 can be configured to measure this distance and computing system 256 can communicate the distance to centralized operating theater controller 302. With some embodiments, laser energy console 246 can be configured to determine the composition and/or texture of the stone 280 and computing system 256 can communicate the composition and/or texture to centralized operating theater controller 302.

In some embodiments, laser energy console 246 can be configured to measure characteristics of the laser energy and treatment environment (e.g., intensity of laser energy, intensity of reflected laser energy, intensity of autofluorescence emitted responsive to incidence of laser energy on the stone, etc.). Processor 132 can execute application instructions 146 to receive this information as physiological information 328a and derive the distance between the distal end 260 and the stone 280, the composition of the stone 280, and/or the texture of the stone 280 based on physiological information 328a.

In some embodiments, processor 132 can execute application instructions 146 to receive physiological information 328a comprising indications of a size of the stone 280 from endoscope 204. For example, processor 132 can execute application instructions 146 to receive captured scene information 330 where the stone 280 is represented. As another example, processor 132 can execute application instructions 146 to receive radiological image physiological information 328a from radiological imaging devices 106 (e.g., an ultrasound, an x-ray, or the like) where the stone 280 is represented in the radiological image physiological information 328a.

Continuing to block 408 “determine other physiological information from the received physiological information” other physiological data can be determined (e.g., derived, inferred, or the like) from physiological information 328a received at block 406. Processor 132 can execute application instructions 146 to cause centralized operating theater controller 302 to derive and/or infer other physiological information 328b from physiological information 328a.

In some embodiments, memory storage device 134 can store models 334. Models 334 can comprise algorithms, functions, and/or trained machine learning (ML) models configured to derive and/or infer physiological information 328b from physiological information 328a.

For example, processor 132 can execute application instructions 146 to determine, using models 334, a turbidity and/or clarity of captured scene information 330 from physiological information 328a and/or captured scene information 330. As another example, processor 132 can execute application instructions 146 to determine, using models 334, a distance between the distal end 260 and the stone 280, a composition of the stone 280, a texture of the stone 280, and/or a size of the stone (e.g., viewed from the camera 218 and/or viewed radiologically) from physiological information 328a, captured scene information 330, and/or radiological image information 332.

Continuing to block 410 “generate a number of graphical elements from the physiological information” several graphical elements 336 can be generated from the physiological information 328a and 328b (including the captured scene information 330 and radiological image information 332). In general, processor 132 can execute application instructions 146 to generate graphical elements 336 representative of physiological information 328a and 328b associated with each component in the OR suite (e.g., endoscope 204, fluidics unit 228, laser energy console 246, etc.) Further, the graphical elements 336 visually depict represented information using images, text, icons, colors, movement, or the like. With some embodiments, the graphical elements 336 can be like “alerts”or pop-up graphics.

For example, processor 132 can execute application instructions 146 to generate graphical elements 336 comprising an indication of the captured scene information 330. As another example, processor 132 can execute application instructions 146 to generate graphical elements 336 comprising an indication of an ILP and/or flow rate. As another example, processor 132 can execute application instructions 146 to generate graphical elements 336 comprising an indication of a size of the stone 280 or a composition of the stone 280. As another example, processor 132 can execute application instructions 146 to generate graphical elements 336 comprising an indication of an intensity of the laser energy incidence on the stone 280, a distance between the distal end 260 and the stone 280, or the like.

In some embodiments, the information visually represented in graphical elements 336 is based on procedure preferences 324. For example, the information is based on the procedure type and equipment provisions, which can be indicated in procedure preferences 324. As a further example, a first physician (e.g., in operating theater 304) may prefer to pay attention to a first subset of the physiological information 328a and 328b while another physician (e.g., in remote room 308) may prefer to pay attention to a second subset of the physiological information 328a and 328b. In general, the first and second subsets may overlap, but this is not required.

Continuing to block 412 “generate, for each room of the OR suite, a room display based on the graphical elements” room displays 338 comprising multiple graphical elements 336 can be generated for each room of OR environment 300. For example, a display of room displays 338 can be generated for theater display 222, remote display 320, and/or observation display 322. As outlined above, each of room displays 338 may comprise different combinations of graphical elements 336 depending upon which physical display (e.g., theater display 222, remote display 320, observation display 322, or the like) the display is to be displayed on. In some embodiments, the processor 132 can execute application instructions 146 to generate room displays 338 from graphical elements 336 based on procedure preferences 324. For example, a user of operating theater 304 viewing theater display 222 can specify which graphical elements 336 are preferred or desired to be visible on theater display 222. These preferences can be dictated in procedure preferences 324 and the processor 132 can execute application instructions 146 to generate a display of room displays 338 for theater display 222 from graphical elements 336 based on the procedure preferences 324 for theater display 222. As another example, a user of remote room 308 viewing remote display 320 can specify which graphical elements 336 are preferred or desired to be visible on remote display 320. These preferences can be dictated in procedure preferences 324 and the processor 132 can execute application instructions 146 to generate a display of room displays 338 for remote display 320 from graphical elements 336 based on the procedure preferences 324 for remote display 320.

Logic flow 400b can begin at block 414. At block 414 “receive a control signal from equipment controls in a remote room of an OR suite” control signals from equipment controls in a remote room of an OR suite can be received. Processor 132 can execute application instructions 146 to cause centralized operating theater controller 302 to receive actuation control signals 340 from equipment controls 314b in remote room 308. For example, during operation, a user in remote room 308 can actuate equipment controls 314b and equipment controls 314b can generate actuation control signals 340, which can be communicated to and received by centralized operating theater controller 302 (e.g., via IT infrastructure 120, or the like).

Continuing to block 416 “process the received control signal” the received control signals can be processed into processed control signals. For example, processor 132 can execute application instructions 146 to process actuation control signals 340 to generate processed control signals 342. In general, processed control signals 342 are versions of actuation control signals 340 configured, processed, or otherwise translated for communication to equipment in operating theater 304 (e.g., robotic devices 108, endoscope 204, endoscope handle 208, fluidics unit 228, laser energy console 246, treatment fiber 254, or the like).

Continuing to block 418 “send the processed controls to equipment in an operating theater of the OR suite” the processed control signals can be sent to equipment in the operating theater of the OR suite associated with the remote room for which the control signals were received. For example, processor 132 can execute application instructions 146 to send processed control signals 342 to equipment in operating theater 304. For example, actuation control signals 340 may be directed to laser energy console 246 and specify actuation or initiation of laser energy generation. As such, processor 132 can execute application instructions 146 to send processed control signals 342 to laser energy console 246 to cause laser energy console 246 to generate laser energy. As another example, procedure preferences 324 may specify a desire for fluid flow at the treatment site. As such, processor 132 can execute application instructions 146 to send processed control signals 342 to fluidics unit 228 to cause fluidics unit 228 to pump (e.g., via pump 238) fluid through cassette and tubing set 242. With some embodiments, blocks block 416 and block 418 can be initiated responsive to receiving a control signal or signals at block 414.

Logic flow 400b can optionally include blocks 420 to block 424. Where logic flow 400b includes these blocks, logic flow 400b can continue from block 418 to block 420. At block 420 “receive feedback from the equipment in the operating theater” feedback signals from equipment in the operating theater of the OR suite can be received. Processor 132 can execute application instructions 146 to cause centralized operating theater controller 302 to receive feedback signals 344 from equipment (e.g., endoscope 204, endoscope handle 208, fluidics unit 228, laser energy console 246, or the like) in operating theater 304.

Continuing to block 422 “process the received feedback” the feedback signals can be processed into processed feedback signals. For example, processor 132 can execute application instructions 146 to process feedback signals 344 to generate processed feedback signals 346. In general, processed feedback signals 346 are versions of feedback signals 344 configured, processed, or otherwise translated for communication to equipment controls 314b in remote room 308.

Continuing to block 424 “send processed feedback to the equipment controls in the remote room” the processed feedback signals can be sent to the equipment controls in the remote room of the OR suite. For example, processor 132 can execute application instructions 146 to send processed feedback signals 346 to equipment controls 314b in remote room 308. With some embodiments, blocks block 422 and block 424 can be initiated responsive to receiving feedback at block 420.

As described herein, an OR suite can include multiple displays. For example, the OR environment 200 described above provisioned for a lithotripsy procedure has at least 4 displays, the main theater display 222, the display 220 for the endoscope 204, the display 236 for the fluidics unit 228, and the display 258 for the laser energy console 246. Further, it is to be appreciated that this does not include patient specific devices (e.g., vital monitoring devices, or the like) and anesthesia devices. To that end, the present disclosure provides an OR suite configured to centralize the display of information relevant to the procedure or therapy with which the OR suite is provisioned.

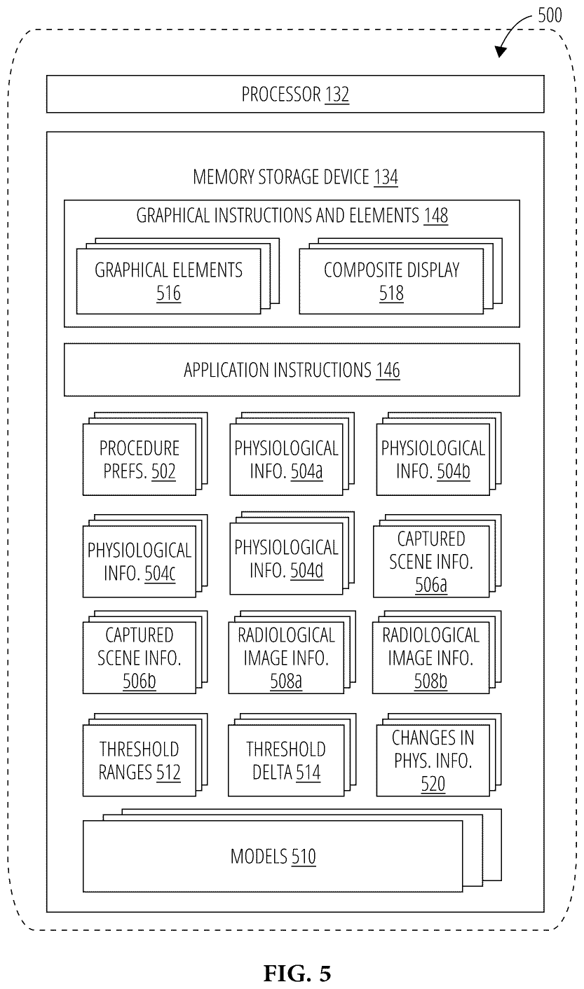

FIG. 5 illustrates a centralized operating theater controller 500, which can be implemented as centralized operating theater controller 122 and/or centralized operating theater controller 302 discussed above. It is to be appreciated that this figure does not depict all elements of centralized operating theater controller 500. For example, elements such as the operating system 144, interconnects, or the like are omitted for clarity. Centralized operating theater controller 500 and particularly an OR suite in which centralized operating theater controller 500 is implemented provides an advantage over conventional OR suites. For example, in each physical location of the OR suite, a user (e.g., a physician) can have a single monitor or display configured to provide relevant information from all or any desired combination of equipment (e.g., imaging devices 106, robotic devices 108, patient devices 110, therapy consoles 112, and/or therapy devices 114) provisioned in the OR suite.

Centralized operating theater controller 500 includes at least a processor 132 and memory storage device 134. In general, memory storage device 134 stores instructions (e.g., application instructions 146, or the like) executable by the processor 132, which when executed cause the centralized operating theater controller 500 to provide a centralized display as described herein. Centralized operating theater controller 500 is described with reference to OR environment 200 and an example lithotripsy procedure for clarity of presentation. However, it is to be appreciated that centralized operating theater controller 500 could be implemented to provide centralized display of information for any of a variety of urological procedures.

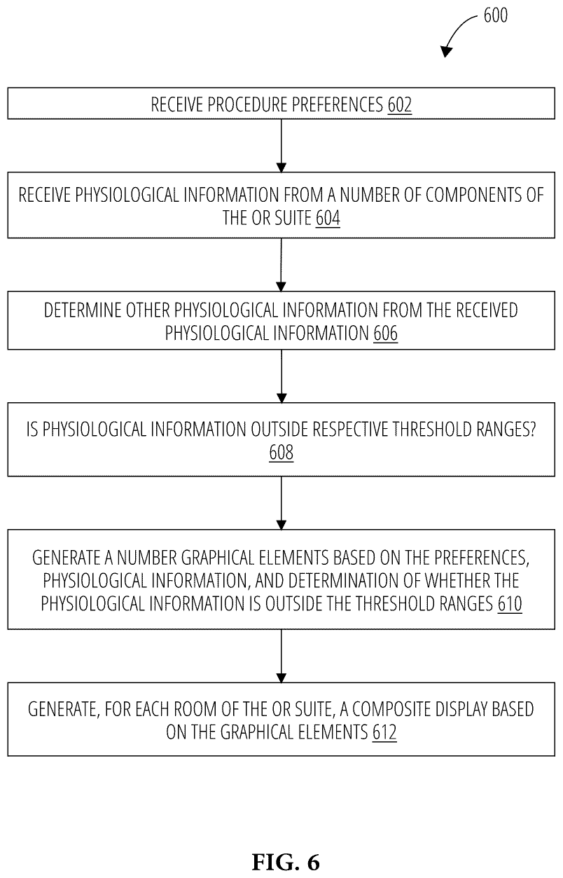

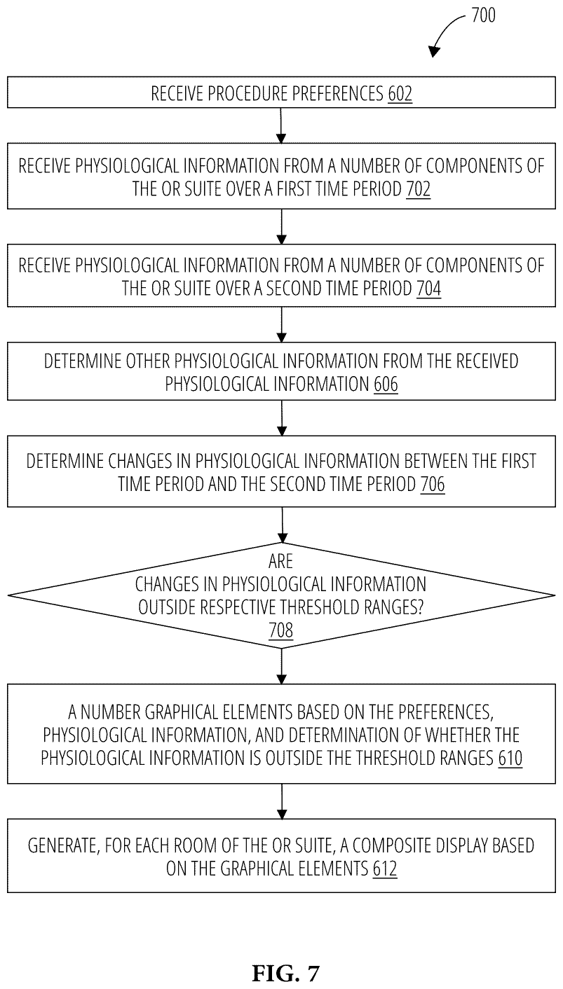

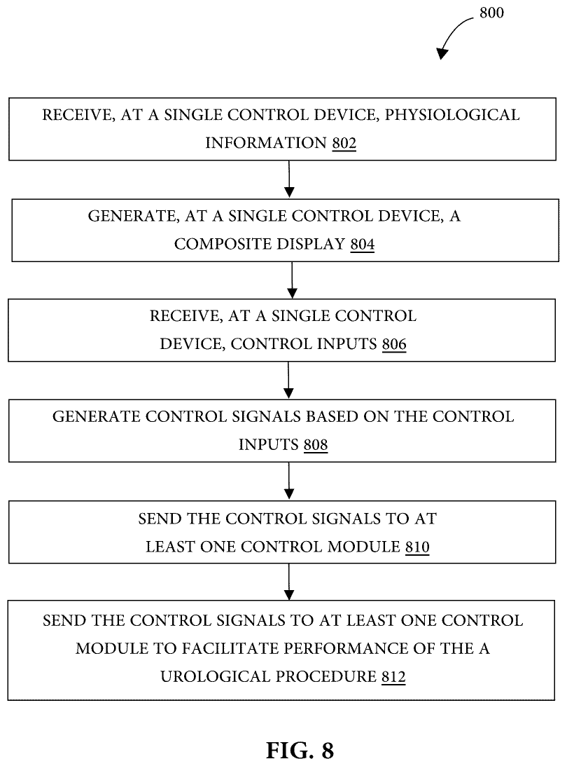

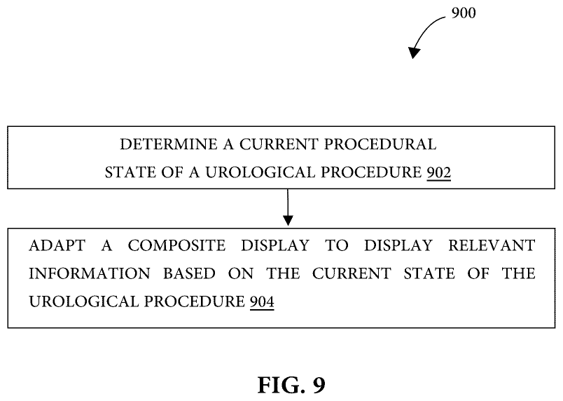

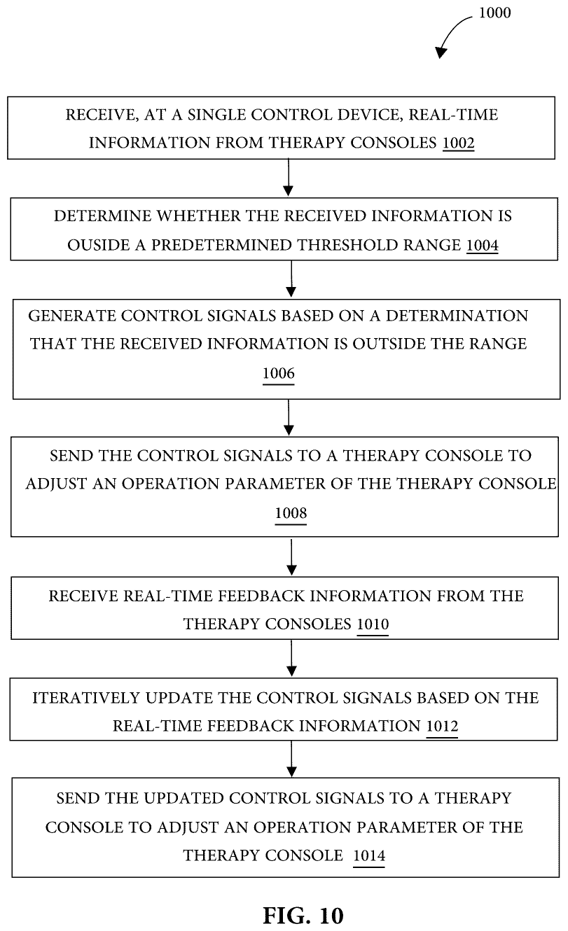

Further, detailed elements of centralized operating theater controller 500 are described with reference to FIGS. 6-10. FIG. 6 illustrates logic flow 600, which can be carried out by centralized operating theater controller 500 to provide a centralized display of information. FIG. 7 illustrates a logic flow 700 which can be carried out by centralized operating theater controller 500 to provide a centralized display of information based on whether physiological information is outside a threshold range. FIG. 8 illustrates a logic flow 800 which can be carried out by centralized operating theater controller 500 to provide centralized control of one or more therapy consoles. FIG. 9 illustrates a logic flow 900 which can be carried out by centralized operating theater controller 500 to adapt the centralized display of information to a current procedural state (e.g., pre-operative planning, intra-operative treatment, or post-operative monitoring). FIG. 10 illustrates a logic flow 1000 which can be carried out the centralized operating theater controller 500 to provide closed loop control of one or more therapy consoles.

Referring to FIG. 6, logic flow 600 can begin at block 602. At block 602 “receive procedure preferences” preferences for the treatment procedure being carried out can be received. In some examples, these preferences can be based on the type of procedure and equipment provisioned in the OR suite. In further examples, these preferences can be based on a physician in the OR suite, established clinic preferences, or the like. Processor 132 can execute application instructions 146 to cause centralized operating theater controller 500 to receive procedure preferences 502 (e.g., from a data center, from local storage, from a cloud-based storage location, or the like). For example, processor 132 can execute application instructions 146 to access remote devices 130 and retrieve procedure preferences 502 associated with the OR suite, procedure, and physician. Procedure preferences 502 are described in greater detail below.

Continuing to block 604 “receive physiological information from a number of components of the OR suite” physiological data can be received from components of the OR environment 200. Processor 132 can execute application instructions 146 to cause centralized operating theater controller 500 to receive physiological information 504a from devices and/or consoles of OR environment 200. For example, processor 132 can execute application instructions 146 to receive data (e.g., an information element, sensor output signals, or the like) comprising indications of an intensity of laser energy (e.g., diagnostic energy, aiming energy, treatment energy, or the like) generated by laser generators 248. With some embodiments, processor 132 can execute application instructions 146 to receive physiological information 504a from computing system 256 of laser energy console 246. With some embodiments, processor 132 can execute application instructions 146 to determine the intensity based on signals received from sensors (not shown) of laser energy console 246 where the sensors are configured to measure qualities and/or characteristics of the laser energy.

In some embodiments, processor 132 can execute application instructions 146 to receive physiological information 504a comprising indications of turbidity and/or clarity of scene(s) captured by camera 218 of endoscope 204. In some embodiments, processor 132 can execute application instructions 146 to receive captured scene information 506a comprising image frames of the scene(s). In some embodiments, processor 132 can execute application instructions 146 to receive physiological information 504a comprising indications of an ILP, flow rate of fluidics unit 228, or both. With some embodiments, information related to multiple scenes can be received (e.g., captured scene information 506a and 506b).

In some embodiments, processor 132 can execute application instructions 146 to receive physiological information 504a comprising indications of a distance between the distal end 260 of the treatment fiber 254 and the stone 280, a composition of the stone 280, and/or a texture of the stone 280. With some embodiments, laser energy console 246 can be configured to measure this distance and computing system 256 can communicate the distance to centralized operating theater controller 500. With some embodiments, laser energy console 246 can be configured to determine the composition and/or texture of the stone 280 and computing system 256 can communicate the composition and/or texture to centralized operating theater controller 500.

In some embodiments, laser energy console 246 can be configured to measure characteristics of the laser energy and treatment environment (e.g., intensity of laser energy, intensity of reflected laser energy, intensity of autofluorescence emitted responsive to incidence of laser energy on the stone, etc.) Processor 132 can execute application instructions 146 to receive this information as physiological information 504a and derive the distance between the distal end 260 and the stone 280, the composition of the stone 280, and/or the texture of the stone 280 based on physiological information 504a.

In some embodiments, processor 132 can execute application instructions 146 to receive physiological information 504a comprising indications of a size of the stone 280 from endoscope 204. For example, processor 132 can execute application instructions 146 to receive captured scene information 506a/506b where the stone 280 is represented. As another example, processor 132 can execute application instructions 146 to receive radiological image information 508a from radiological imaging devices 106 (e.g., an ultrasound, an x-ray, or the like) where the stone 280 is represented in the radiological image information 508a. With some embodiments, information related to multiple radiological images can be received (e.g., radiological image information 508a and 508b).

Continuing to block 606 “determine other physiological information from the received physiological information” other physiological data can be determined (e.g., derived, inferred, or the like) from physiological data received at block 604. Processor 132 can execute application instructions 146 to cause centralized operating theater controller 500 to derive and/or infer other physiological information 504b from physiological information 504a.

In some embodiments, memory storage device 134 can store models 510. Models 510 can comprise algorithms, functions, and/or trained machine learning (ML) models configured to derive and/or infer physiological information 504b from physiological information 504a.

For example, processor 132 can execute application instructions 146 to determine, using models 510, a turbidity and/or clarity of captured scene information 506a/506b from physiological information 504a and/or captured scene information 506a/506b. As another example, processor 132 can execute application instructions 146 to determine, using models 510, a distance between the distal end 260 and the stone 280, a composition of the stone 280, a texture of the stone 280, and/or a size of the stone (e.g., viewed from the camera 218 and/or viewed radiologically) from physiological information 504a, captured scene information 506a/506b, and/or radiological image information 508a/508b.

Continuing to block 608 “is physiological information outside respective threshold ranges?” a determination of whether the physiological information is outside respective threshold ranges is made. Processor 132 can execute application instructions 146 to determine whether physiological information 504a and 504b are outside (or within) threshold ranges 512 and store the determinations as threshold delta 514 in memory storage device 134. With some embodiments, threshold ranges 512 can be set at the factory by the medical device manufacturer of the component. In other embodiments, threshold ranges 512 can be configured by a user (e.g., as part of procedure preferences 502, or the like). In yet other embodiments, threshold ranges 512 can be dynamically set based on physiological information 504a and 504b. That is, a range of threshold ranges 512 for one value of physiological information 504a or 504b can be dynamically set (e.g., during a procedure) based on other values of physiological information 504a and/or 504b. Accordingly, threshold ranges 512 can include any combination of pre-set thresholds, configurable thresholds, and dynamically set thresholds. As used herein, the term range is to mean either a range bound of both ends (e.g., between 0 and 1) and a range bound on only one end (e.g., less than or equal to 0 or greater than or equal to zero). Accordingly, as used herein the term “outside” the threshold range is intended to mean outside the range (e.g., less than the lower bound or greater than the upper bound, where bound on both ends, less than the bound when bound on a lower end, or greater than the bound when bound on a higher end).

In other examples, the term threshold range is used to mean a specific value or characteristic of physiological information 504a and/or 504b (e.g., composition, texture type, or the like). Further, in some embodiments, multiple thresholds can be specified for some values and/or characteristics of physiological information 504a and/or 504b.

For example, processor 132 can execute application instructions 146 to determine whether a composition of the stone 280 (as indicated in physiological information 504a and/or 504b) is outside (e.g., different from) a composition specified by threshold ranges 512. As another example, processor 132 can execute application instructions 146 to determine whether the size of the stone 280 (as indicated in physiological information 504a and/or 504b) is less than a threshold size (e.g., passable size, size small enough to retrieve, or the like). As another example, processor 132 can execute application instructions 146 to determine whether the distance between the distal end 260 of the treatment fiber 254 and the stone 280 (as indicated in physiological information 504a and/or 504b) is greater than a threshold distance. With some examples, there are multiple thresholds provided for the distance between the distal end 260 and the stone 280 in threshold ranges 512. In such an example, processor 132 can execute application instructions 146 to determine which, if any, threshold the distance is outside of.

As another example, processor 132 can execute application instructions 146 to determine whether a turbidity and/or clarity (as indicated in physiological information 504a and/or 504b) of the captured scene information 506a/506b is outside that specified by threshold ranges 512. As another example, processor 132 can execute application instructions 146 to determine whether the image saturation (as indicated in physiological information 504a and/or 504b) of the captured scene information 506a/506b is outside that specified by threshold ranges 512. In yet another example, processor 132 can execute application instructions 146 to determine whether the visibility of an aiming beam for the laser energy (as indicated in physiological information 504a and/or 504b) of the captured scene information 506a/506b is outside that specified by threshold ranges 512.

Continuing to block 610 “generate a number of graphical elements from the physiological information and the determination of whether the physiological information is outside the threshold ranges based on the preferences” several graphical elements 516 can be generated from the physiological information 504a and 504b (including the captured scene information 506a/506b and radiological image information 508a/508b) and the threshold delta 514 based on the procedure preferences 502. In general, processor 132 can execute application instructions 146 to generate graphical elements 516 representative of physiological information 504a and 504b associated with each component in the OR suite (e.g., endoscope 204, fluidics unit 228, laser energy console 246, etc.) Further, the graphical elements 516 visually depict represented information using images, text, icons, colors, movement, or the like. With some embodiments, the graphical elements 516 can be like “alerts”or pop-up graphics.

For example, processor 132 can execute application instructions 146 to generate graphical elements 516 comprising an indication of the captured scene information 506a/506b. As another example, processor 132 can execute application instructions 146 to generate graphical elements 516 comprising an indication of an ILP and/or flow rate. As another example, processor 132 can execute application instructions 146 to generate graphical elements 516 comprising an indication of a size of the stone 280 or a composition of the stone 280. As another example, processor 132 can execute application instructions 146 to generate graphical elements 516 comprising an indication of an intensity of the laser energy incidence of the stone 280, a distance between the distal end 260 and the stone 280, or the like.

In some embodiments, the information visually represented in graphical elements 516 is based on procedure preferences 502. For example, the information is based on the procedure type and equipment provisions, which is indicated in procedure preferences 502. As a further example, a first physician may prefer to pay attention to a first subset of physiological information 504a and 504b while another physician may prefer to pay attention to a second subset of physiological information 504a and 504b. In general, the first and second subsets may overlap, but this is not required.

Continuing to block 612 “generate, for each room of the OR suite, a composite display based on the graphical elements” composite displays 518 comprising multiple graphical elements 516 can be generated for each room or geographic location of the OR suite. For example, a composite displays 518 can be generated for each internal operating room display 102 or external operating room display 104. As outlined above, each composite displays 518 may comprise different combinations of graphical elements 516 depending upon which physical display (e.g., theater display 222, or the like) the composite displays 518 is to be displayed on.

In some examples, graphical elements 516 can be generated based on a change in physiological information 504a and/or 504b. FIG. 7 illustrates logic flow 700, which can be carried out by centralized operating theater controller 500 to provide a centralized display of information. Logic flow 700 is much like logic flow 600 and where operations overlap, logical actions from logic flow 600 are reused in logic flow 700.

Logic flow 700, like logic flow 600, can begin with block 602. Continuing to block 702 “receive physiological information from a number of components of the OR suite over a first time period” and then to block 704 “receive physiological information from a number of components of the OR suite over a first time period” physiological data can be received from components of the OR environment 200 during a first time period (e.g., at block 702) and a second time period (e.g., at block 704). Blocks 702 and 704 of logic flow 700 are like block 604 of logic flow 600, except that blocks 702 and 704 receive physiological information 504a and 504c in respective first and second time periods. In some embodiments, the second time period is after the first time period. With some embodiments, the first and the second time periods can be snapshots in time separated by a time duration (e.g., 1 second(s), 0.5 s, 0.1 s, 0.01 s, 0.001 s, 0.0001 s, between 0.5 s and 1 s, or between 0.001 s and 0.1 s).

Logic flow 700 continues with block 606 from logic flow 600. However, as there is physiological information from two time periods (e.g., physiological information 504a and 504c), processor 132 can execute application instructions 146 to cause centralized operating theater controller 500 to derive and/or infer other physiological information 504b and 504d from physiological information 504a and 504c, respectively.

Continuing to block 706 “determine changes in physiological information between the first time period and the second time period” changes in physiological information from the first time period to the second time period can be determined. For example, processor 132 can execute application instructions 146 to determine changes in physiological information 520 based on the difference between physiological information 504a and 504c and physiological information 504b and 504d. As a specific example, processor 132 can execute application instructions 146 to determine a change in the size of stone 280 from the first time period (e.g., physiological information 504a and 504c) to the second time period (e.g., physiological information 504b and 504d). As another example, processor 132 can execute application instructions 146 to determine a change in the composition of the stone 280 from the first time period (e.g., physiological information 504a and 504c) to the second time period (e.g., physiological information 504b and 504d). As another example, processor 132 can execute application instructions 146 to determine a motion of the stone 280 between the first time period (e.g., physiological information 504a and 504c) and the second time period (e.g., physiological information 504b and 504d).

Continuing to decision block 708 “are changes in physiological information outside respective threshold ranges?” a determination of whether the changes in physiological information from the first time period to the second time period are outside respective threshold ranges is made. Decision block 708 of logic flow 700 can be like block 608 of logic flow 600 except that in logic flow 700, the determination is made with respect to changes in physiological information 520 and not the physiological information itself.

Logic flow 700 can continue with blocks 610 and 612 where graphical elements 516 and composite displays 518 can be generated. In some embodiments, centralized operating theater controller 500 can implement both logic flow 600 and logic flow 700 simultaneously.

FIG. 8 illustrates a fifth logic flow 800 in accordance with at least one embodiment. More specifically, FIG. 8 illustrates a logic flow 800 and associated system implementations for centralized control and display in a urological operating room in accordance with at least one embodiment. The logic flow 800 and corresponding system architectures provide comprehensive functionality for single device control and situational display adaptation during urological procedures across multiple implementation formats including method, system, and computer-readable storage medium embodiments, as detailed herein.

As used herein, a “single control device” refers to a centralized control interface that provides unified operation and display capabilities for multiple therapy consoles in a urological operating room environment. For instance, the “single control device” can include a centralized controller, such as the centralized operating theater controller 500 illustrated in FIG. 5, that is configured to provide unified operation and display capabilities for multiple therapy consoles in a urological operating room environment.

Specific examples of the single control device include mobile computing devices such as mobile phones, tablets, or laptops that are located remote to the therapy consoles, providing portability and flexibility for users both within and outside the sterile field. The single control device can additionally or alternatively be implemented as fixed displays within the operating environment, including operating theater displays that are visible to multiple observers in the surgical suite, or observation room displays positioned outside the operating theater for monitoring by support staff or remote specialists. That is, one or more devices such as a tablet and/or a built-in monitor or display located in another device in or associated with the OR can be configured as the single control device. As mentioned, in some embodiments, the single control device can be a wearable device. For example, the physician or other individual can wear a head-mounted display or other type wearable display. In some embodiments, internal operating room display 102 can include an on-patient display, for example, a display or monitor physically attached to the patient, a display projected onto the patient, or the like that can be configured as the single control device.

The single control device can include a display (e.g., a touch display) and/or various input mechanisms in combination with a display (e.g., a touch display, etc.). In some embodiments, the single control device can be configured to receive voice commands or other types of inputs. For instance, having the single control device be configured to receive voice commands can desirably allow for hands-free or minimal-contact operation during sterile procedures.

At block 802, the logic flow 800 can include “RECEIVING, AT A SINGLE CONTROL DEVICE, PHYSIOLOGICAL INFORMATION FROM A PLURALITY OF THERAPY CONSOLES PROVISIONED IN AN OPERATING THEATER”. For instance, the single control device can be configured to receive real-time operational data from at least a subset (e.g., those involved in a particular type of medical procedure) of the plurality of therapy consoles. As detailed herein, the physiological information may comprise one or more of pressure measurements, fluid flow parameters, energy settings, power levels, captured scene information, and/or radiological image information, among other types of physiological information. As mentioned, the plurality of therapy consoles may include at least a fluidics unit, a laser energy console, and an endoscope, each configured to provide specific procedural data. In some embodiments, the received information may further include turbidity measurements, clarity assessments, temperature readings, procedure state indicators, and/or target tissue state information derived from the primary physiological data, among other types of received information.

At block 804, the logic flow 800 can include, “generating, at the single control device, a composite display comprising combined visual indications from multiple ones of the plurality of therapy consoles”. That is, the logic flow 800 can create unified visual presentations in the form of a composite display. The composite display integrates multiple graphical elements representing visual indications of the physiological information from various therapy consoles simultaneously. In some embodiments, the combined visual indications may be representative of physiological information, captured scene information, radiological image information, or any combination thereof. The combined visual information can be presented through a single interface (e.g., in the single control device) that consolidates operational parameters and/or other information from and/or information derived from each of the therapy consoles.

The composite display generation is implemented through processor-executable instructions that when executed by processors of centralized control devices cause the generation of unified visual presentations, and through system architectures that include dedicated display components configured to show composite visual information from multiple therapy console sources.

At block 806, the logic flow 800 can include, “receiving, at the single control device, control inputs from a user”. That is, the single control device can be configured to accept user commands during a urological procedure, such as those described herein, for procedural control of one or more of the plurality of therapy consoles and/or other components (e.g., lighting in the OR, etc.). The control inputs may be received through various interface mechanisms including mobile computing devices located remote to the therapy consoles, such as mobile phones, tablets, or laptops, or through operating theater displays or observation room displays.

At block 808, the logic flow 800 can include “generating control signals based on the control inputs”. That is, the systems herein can process user commands into executable instructions. For instance, the single control device can be configured to process the received user commands into control signals. Alternatively, or additionally, the control signals can be generated via a feedback loop (e.g., a closed feedback loop), as detailed herein with respect to FIG. 10.

The control signals can be sent to one or more devices such as one or more therapy consoles. In some embodiments, the control signals may comprise coordinated control commands that cause synchronized operation between multiple therapy consoles. As mentioned, in some embodiments the control signals can be generated based on one or more user inputs (e.g., user inputs provided to the single control device). However, in some embodiments, the generation process may incorporate the determined procedural state and type to optimize control signal parameters. Hence, in some embodiments the system may automatically generate different control signals based on any procedural preferences and at least the current state of the urological procedure, adapting the control approach for pre-operative planning, intra-operative treatment, or post-operative monitoring phases, as detailed herein.

Similarly, in some embodiments the system may automatically generate different control signals based on a deviation (e.g., a magnitude, a positive or negative value of deviation, and/or a duration of the deviation) from a predetermined threshold range. For instance, as detailed with respect to FIG. 10, the control signals can be generated to mitigate (e.g., reduce a magnitude of an absolute value of a deviation from a predetermined threshold range).

At block 810, the logic flow 800 can include, “sending the control signals from the single control device to multiple ones of the plurality of therapy consoles to provide real-time coordinated control during performance of the urological procedure”. The coordinated control may include adjustment of one or more therapy console parameters such as pressure settings, fluid flow rates, laser energy parameters, and visualization settings through the composite display. The one or more therapy console parameters may be associated with one or more therapy consoles.

In some embodiments, the system may receive a feedback signal from one or more of the therapy consoles responsive to the control signals (received by at least one therapy console) and update the composite display based on the feedback information from the therapy consoles to reflect real-time status changes across all therapy consoles, thereby providing real-time feedback to users. Alternatively, or in addition, in some embodiments the system (e.g., the single control device) can be configured to automatically generate an updated control signal based on the real-time feedback information, as detailed herein with respect to FIG. 10. That is, the systems herein can automatically generate controls signals to mitigate (e.g., reduce or eliminate) a deviation from the minimum or maximum value of the predetermined threshold range). For example, the control signals can be generated and sent to one or more therapy consoles to alter a function of the one or more therapy consoles. Hence, subsequently received information (e.g., real-time feedback information) from the one or more therapy consoles and/or other therapy consoles can be within or at least closer to a predetermined threshold range associated with the received information.

The logic flow 800 encompasses comprehensive system implementations including urological operating room control systems comprising operating theaters with provisioned therapy consoles, single control devices with integrated display and input capabilities, and processors configured to execute the full range of centralized control and display operations. That is, the logic flow 800 supports both the fundamental centralized control concept where single control devices provide centralized display of information from all therapy consoles and centralized control of all therapy consoles through composite displays, and the advanced situational display functionality where composite displays adapt based on real-time feedback and/or determined procedural states to provide contextually appropriate information for each procedural phase, facilitating unified operation of complex urological OR environments while reducing cognitive and operational burdens on medical professionals across multiple implementation formats and system architectures.

FIG. 9 illustrates a sixth logic flow 900 for procedural state determination and adaptive display control in a urological operating room in accordance with at least one embodiment. The logic flow 900 addresses at least the technical challenge of providing contextually appropriate information displays that automatically adapt based on the current phase of urological procedures, thereby reducing cognitive burden on medical professionals while ensuring relevant information is prominently presented during each procedural phase.

Urological procedures typically progress through distinct procedural states, each characterized by different informational requirements and operational priorities that necessitate adaptive display and control configurations. The system recognizes three primary procedural states that define the operational context for urological procedures: pre-operative planning, intra-operative treatment, and post-operative monitoring. Pre-operative planning encompasses the preparatory phase where procedural strategy is established, equipment configurations are determined, imaging studies are reviewed, and treatment parameters are set based on patient-specific factors and procedural requirements. Intra-operative treatment represents the active treatment phase during which therapeutic interventions are performed, real-time physiological monitoring occurs, dynamic parameter adjustments are implemented, and immediate procedural decisions are made based on evolving conditions within the urological anatomy. Post-operative monitoring constitutes the concluding phase where treatment outcomes are assessed, procedural efficacy is evaluated, patient status is monitored for complications, and documentation of procedural results is completed. The determination of the current procedural state permits the systems herein to automatically adapt composite displays, prioritize relevant information, and optimize control configurations to match the specific informational and operational needs of each procedural phase, thereby enhancing procedural efficiency and reducing cognitive burden on medical professionals throughout the urological procedure continuum.

At block 902, “determining a current procedural state of a urological procedure,” the centralized operating theater controller analyzes multiple technical indicators and data inputs to establish the current phase of the urological procedure through comprehensive state transition detection mechanisms. The procedural state determination encompasses identifying whether the current phase comprises one of pre-operative planning for the urological procedure, intra-operative treatment during the urological procedure, or post-operative monitoring after the urological procedure.

For instance, the systems herein can detect procedural state transitions such as a transition from pre-operative planning to intra-operative treatment through analysis of equipment activation signals including laser energy console activation status indicating commencement of active treatment, endoscope insertion detection through pressure sensor activation, and/or fluidics unit pump engagement indicating irrigation initiation. Physiological parameter changes serve as alternate or additional state transition indicators. Physiological parameter changes can include initial intraluminal pressure measurements indicating scope insertion, temperature sensor activation at distal treatment device ends, and captured scene information transitions from ambient lighting to internal anatomical visualization. The systems herein can also monitor user input signals including foot pedal activation for laser energy delivery, endoscope handle control engagement, and/or therapy console parameter adjustments which can be indicative of a current procedural state such as indicating active treatment initiation or an absence of which can be indicative of a pre-operative planning state or post-operative state.

In some embodiments, the systems herein can identify intra-operative to post-operative transitions through detection of treatment completion indicators including laser energy console shutdown following treatment completion, sustained reduction in fluidics flow rates indicating irrigation cessation, endoscope withdrawal detection through pressure sensor deactivation, stone fragmentation completion detection through captured scene analysis, target tissue treatment confirmation through visual assessment algorithms, and/or procedural goal achievement verification through imaging analysis. Additionally, the systems herein can also monitor user input signals including foot pedal activation for laser energy delivery, endoscope handle control engagement, and/or therapy console parameter adjustments which can be indicative of a current procedural state such as indicating active treatment initiation or an absence of which can be indicative of another state (e.g., post-operative state).

In some embodiments, physiological parameter stabilization can provide further transition confirmation, including intraluminal pressure normalization to baseline levels, temperature equilibration to physiological ranges, and fluid balance calculations indicating procedural completion. Additionally, the systems herein can determine the type of the urological procedure being performed. That is, the systems herein can be tailored to a particular type of urological procedure recognizing that different procedural categories such as stone treatment procedures, soft tissue treatment procedures, diagnostic procedures, or therapeutic interventions, which may influence the specific information prioritization and display organization for each procedural phase of the particular type of urological procedure.

In some embodiments, the logic flow 900 can adapt the composite display to display a first composite display including relevant information based on the type of the urological procedure, the procedural preferences associated with the urological procedure, the current state of the urological procedure, or any combination thereof. For instance, at block 904, “adapt a composite display to display relevant information based on the current state of the urological procedure”, the systems herein can automatically implement comprehensive system reconfigurations to optimize information presentation and control functionality for each procedural phase. For instance, upon detection of procedural state changes, display adaptation can occur (e.g., immediately or after elapse of a predetermined time interval) following state transition detection. Hence, the systems herein can automatically alter composite displays to present information sets appropriate for the current procedural state, prioritizes graphical elements relevant to the active phase, and/or modifies user interface configurations to emphasize critical parameters for the current operational context.

In some embodiments, control parameter adjustments can accompany display modification (e.g., modification associated with a current procedural state). Control parameter adjustments can include automatic reconfiguration of threshold ranges appropriate for the current procedural phase, adjustment of alert and notification parameters based on state-specific requirements, and/or modification of control signal processing algorithms to optimize responsiveness for current operational demands.

Display modification associated with a current procedural state can include modification of the display during one or more of or each of a plurality of determined procedural states. For example, during pre-operative planning, a first composite display can be configured to display (e.g., emphasize) procedural setup information, equipment configuration status, pre-operative imaging data, planned treatment parameters, procedural preferences, and/or equipment configuration settings. However, during intra-operative treatment, the composite display can be configured to automatically transition to display different information e.g., view a second composite display, a third composite display, etc., which are displayed subsequent to the first composite display. For instance, during intra-operative treatment the display can be reconfigured to display and thereby prioritize real-time physiological information including intraluminal pressure, laser energy settings, captured scene information from endoscope cameras, active treatment parameters, visual feeds from therapy devices, and/or immediate procedural feedback data. Moreover, during post-operative treatment the display can display different information than is displayed during the pre-operative planning and during intra-operative treatment. For instance, during post-operative monitoring, the display can automatically reconfigure to focus on treatment outcome indicators, procedural completion status, final pressure readings, treatment outcome assessments, post-treatment evaluations, treatment efficacy indicators, and/or patient status information.

The systems herein can employ automatic state transition detection through specific technical scenarios, such as simultaneous laser console activation, endoscope pressure sensor engagement, and fluidics pump initialization triggering transition from pre-operative displays to real-time operational parameters, followed by automatic reconfiguration to post-operative metrics upon laser deactivation, sustained pressure reduction indicating scope withdrawal, and achievement of predetermined treatment endpoints. The relevant information displayed via the composite display for each procedural state differs significantly, with seamless transitions that maintain continuity of critical information while adapting the interface to evolving procedural requirements throughout the duration of urological procedures.

In some embodiments, the systems herein can verify a current type of procedure and/or verify a current procedural state. For instance, in some embodiments, a current type of procedure, a current procedural state, and/or an occurrence of a procedural state transition can be verified based on temporal analysis algorithms and/or multi-parameter correlation analysis to ensure accurate state detection while preventing false positives (e.g., false positive procedural state changes) such as those that may otherwise be attributable to sensor variation and/or momentary (e.g., inadvertent) equipment adjustments. That is, the systems herein can implement multiple verification mechanisms to ensure accurate state transition detection and prevent false positive state changes, including temporal analysis algorithms (e.g., which can be trained on typical procedural times associated with a given type of urological procedure and/or times associated with one or more procedural states of the given urological procedure) requiring sustained indicator patterns over predetermined time periods to confirm legitimate state transitions, multi-parameter correlation analysis verifying consistent patterns across multiple therapy consoles and monitoring systems. Alternatively, or in addition, the systems herein can employ user confirmation protocols providing additional verification pathways for manual confirmation or override of automatic state transition determinations when clinical circumstances warrant intervention. In short, the systems herein can be configured to provide continuous procedural awareness and reduce cognitive burden on medical professionals.

FIG. 10 illustrates a seventh logic flow in accordance with at least one embodiment. More specifically, FIG. 10 is directed to logic flow 1000 which includes all elements related to the concept of closed loop control which provides comprehensive functionality for automatic monitoring, control signal generation, and iterative feedback-based adjustment of therapy console operations during urological procedures based on real-time information analysis and threshold determinations.

Closed loop control, in the context of the urological operating room system, refers to an automated control methodology where the centralized operating theater controller continuously monitors real-time information from multiple therapy consoles. The centralized operating theater controller can compare this information against predetermined threshold parameters, automatically generates and sends control signals to adjust operational parameters of therapy consoles when threshold deviations are detected, receive feedback information responsive to these adjustments, and iteratively refine the control signals based on the feedback to maintain optimal procedural conditions within desired threshold ranges. Hence, closed loop control represents a significant advancement over the conventional manual control systems where each piece of equipment typically has its own custom computing hardware configuration and display requiring individual monitoring and control. The closed loop control herein can utilize the centralized operating theater controller architecture described herein, to provide automated, real-time parameter adjustment based on continuous feedback from multiple therapy consoles during urological procedures.

The closed loop control can be employed in various practical applications that address specific urological procedure requirements. For example, in the context of intraluminal pressure management, the systems herein can automatically monitor intraluminal pressure readings from pressure sensors integrated into endoscopes or separate pressure sensor devices. In such instances, when a monitored pressure exceeds a safe threshold, the systems herein can generate and send control signals to automatically adjusts a therapy console parameter (e.g., a fluidics unit settings including inflow rates, outflow parameters, and/or pump pressure, etc.) to maintain pressure within predetermined ranges, thereby improving procedural outcomes by controlling intraluminal pressure at the operating site.

Similarly, for turbidity-based irrigation control, the systems herein can monitor turbidity and clarity measurements (e.g., from captured scene information obtained through visualization equipment). In instances when turbidity levels exceed optimal visualization thresholds, the systems herein can automatically alter (e.g., increase) irrigation flow rates through fluidics units to clear the visual field. In such instances, the systems herein can also simultaneously adjust parameters of one or more additional therapy consoles such as adjusting laser parameters to prevent excessive debris generation that could further impair visibility.

At block 1012, “iteratively updating the control signals based on the real-time feedback information,” the systems herein can generate additional control signals based on observed system responses and continuing threshold assessments and/or threshold adjustments. The iterative updating process analyzes feedback effectiveness, calculates remaining deviations, assesses system stability, and determines whether additional control actions (e.g., which can be facilitated via updated control signal generation) are required to achieve optimal operational status. The updating methodology may incorporate predictive algorithms, trend analysis, and/or adaptive control strategies that improve system responsiveness and accuracy through successive iteration cycles. For instance, the updated control signals can be configured to mitigate any remaining deviation from one or more threshold values and/or threshold ranges.

At block 1014, “sending the updated control signals to automatically adjust an operational parameter of the at least one therapy console and mitigate a remaining deviation from the predetermined threshold range,” the systems herein can implement refined control actions (e.g., transmit updated control signals) to address residual threshold exceedances and optimize overall system performance. The updated control signals represent refined parameter adjustments based on observed system responses and continuing feedback analysis, providing progressive optimization toward target operational states. This iterative refinement process continues until threshold compliance is achieved or procedural completion occurs, ensuring continuous optimization of therapy console operations throughout urological procedures while maintaining safety parameters and procedural effectiveness.

The logic flow 1000 implements comprehensive closed loop control functionality that significantly advances urological operating room automation by providing continuous, automatic optimization of therapy console operations based on real-time monitoring and feedback-driven parameter adjustment, thereby reducing manual intervention requirements while improving procedural consistency and outcomes through systematic threshold management and responsive control signal generation. One or more aspects of the logic flow 1000 can be repeated (e.g., iteratively repeated) to promote closed loop control, as detailed herein.

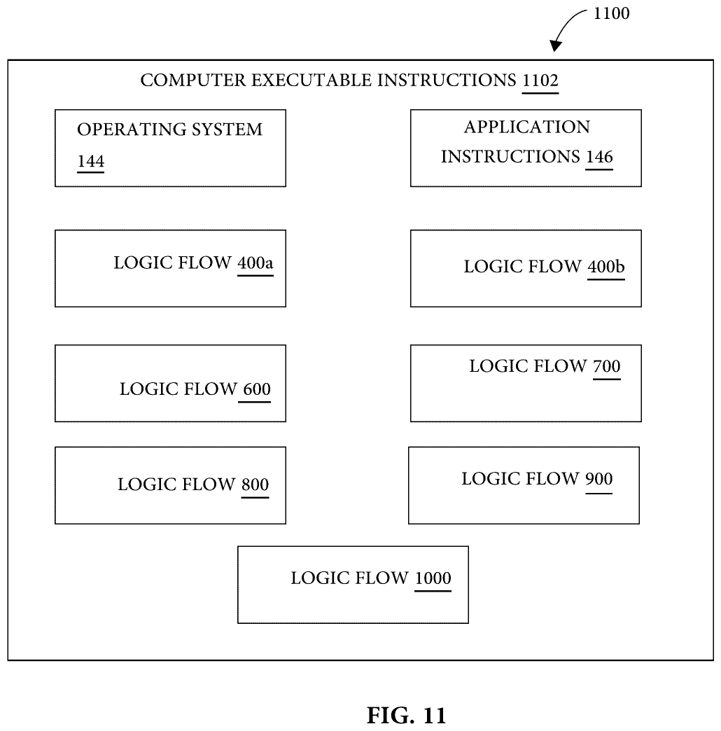

FIG. 11 illustrates computer-readable storage medium 1100. Computer-readable storage medium 1100 may comprise any non-transitory computer-readable storage medium or machine-readable storage medium, such as an optical, magnetic or semiconductor storage medium. In various embodiments, computer-readable storage medium 1100 may comprise an article of manufacture. In some embodiments, computer-readable storage medium 1100 may store computer executable instructions 1102 with which circuitry (e.g., processor 132, or the like) can execute. For example, computer executable instructions 1102 can include instructions to implement operations described with respect to operating system 144, application instructions 146, logic flow 400a, logic flow 400b, logic flow 600, logic flow 700, logic flow 800, logic flow 900, and/or logic flow 1000. Examples of computer-readable storage medium 1100 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1102 may include any suitable type of code, such as source code, compiled code, interpreted code, executable code, static code, dynamic code, object-oriented code, visual code, and the like.

Terms used herein should be accorded their ordinary meaning in the relevant arts, or the meaning indicated by their use in context, unless an express definition is provided, in which case the definition provided herein controls. Additionally, references to “one embodiment” or “an embodiment” do not necessarily refer to the same embodiment. Further, embodiments can be combined where combination does not conflict with the context provided. Words “comprise,” “comprising,” and the like are to be construed in an inclusive sense as opposed to an exclusive or exhaustive sense; that is to say, in the sense of “including, but not limited to. ” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application.

When the claims use the word “or” in reference to a list of two or more items, that word covers all the following interpretations of the word: any of the items in the list, all the items in the list, and any combination of the items in the list, unless expressly limited to one or the other. When the claims use the word “and/or” in reference to a list of two or more items, that word covers any combination of the listed items. For example, where a claim recites “item 1, item 2, and/or item 3,” the claim means item 1 alone, item 2 alone, item 3 alone, items 1 and 2, items 1 and 3, items 2 and 3, or items 1, 2, and 3.

Temperature-controlled therapy delivery represents another application where the systems herein can monitor (e.g., continuously monitor) intraluminal temperature such as a temperature at a treatment site. In such instances, the systems herein can automatically generate and send control signal configured to adjust laser energy parameters, pulse duration, and/or cooling fluid flow rates to prevent thermal damage (e.g., maintain a temperature withing a predetermined temperature range) to surrounding tissues while maintaining therapeutic effectiveness.

In some embodiments, distance-based laser and scope coordination can employ closed loop control. The systems herein can automatically generate and send control signals configured to maintain optimal fiber-to-stone distances by coordinating scope deflection, fiber positioning, and laser energy parameters. This closed loop approach assists treatment by integrating control and feedback of all devices used during the procedure. The coordinated control maintains consistent laser fiber to stone distance and prevents losing track of the stone location throughout the treatment process.

In some embodiments, stone composition adaptive closed loop control can be employed. For instance, the systems herein can analyze stone composition and texture information derived from laser-tissue interaction feedback and automatically adjust laser energy parameters, pulse patterns, and/or fragmentation strategies to optimize stone treatment efficiency based on the determined stone characteristics, supporting the goal to improve stone fragmentation and dusting rates during laser lithotripsy.

In some embodiments, the systems herein can generate and send control signals to multiple therapy consoles substantially simultaneously to control or alter parameters associated with each of the multiple therapy consoles. Such multi-parameter coordination showcases the closed loop control capability of the systems herein to simultaneously manage multiple physiological parameters by coordinating adjustments across different therapy consoles, such as automatically reducing laser power when pressure thresholds are approached, while increasing irrigation flow to maintain visualization.

This closed loop control framework significantly enhances the centralized operating theater controller concept from the provisional application by adding automated response capabilities that reduce the cognitive burden on medical professionals while improving procedural safety and effectiveness through real-time parameter optimization based on continuous feedback from multiple therapy console systems

With continued reference to FIG. 10, the example logic flow 1000 can be begin at block 1002. At block 1002, “receiving, at a single control device, real-time information from a plurality of therapy consoles provisioned in an operating theater”, the systems herein (e.g., the centralized operating theater controller) can receive one or more types of real-time information associated with the urological procedure. The received information can comprise physiological information, captured scene information, radiological image information, procedural state information, or any combination thereof. The physiological information may comprise intraluminal pressure measurements, intraluminal temperature readings, fluid flow rates, laser energy parameters, distance measurements between treatment devices and target tissues, stone composition analysis, and texture determinations. The captured scene information includes turbidity assessments, clarity measurements, image saturation levels, laser aiming beam visibility indicators, and visual scene transitions during procedural phases. Radiological image information includes pre-operative imaging data, intra-operative fluoroscopy results, ultrasound measurements, and multi-modality imaging studies that provide anatomical and positional reference data. Procedural state information includes equipment activation status, time elapsed in procedure phases, treatment efficacy indicators, and operational state transitions that define current procedural context.

At block 1004, “determining whether the received information is outside a predetermined threshold range,” the centralized operating theater controller can be configured to analyze various received real-time information to assess a current operational status against established predetermined thresholds and/or threshold ranges. For instance, each of the types of received information can have a corresponding predetermined threshold (e.g., one or more physiological thresholds, one or more captured scene information thresholds such as a turbidity threshold, etc., one or more radiological thresholds, and/or one or more procedural state thresholds.). The predetermined thresholds can be an individual value (e.g., a minimum acceptable value or a maximum acceptable value) or can be manifested as a threshold range (e.g., including a minimum acceptable and maximum acceptable value).

The predetermined threshold ranges can be configured based on safety parameters, procedural requirements, equipment specifications, and/or clinical protocols. In some embodiments, the ranges of the predetermined threshold ranges can be dynamically adjusted based on procedural type, patient-specific factors, and/or real-time procedural conditions.

Responsive to the determination at 1004, the logic flow 1000 can proceed to block 1006. At block 1006, “automatically generating control signals based on a determination that the received information is outside the predetermined threshold range,” the systems herein can process threshold exceedance determinations into actionable control commands. The automatic generation process utilizes algorithms, decision trees, and/or processing logic to convert threshold deviation measurements into specific control parameters tailored for individual therapy console requirements. Control signal generation incorporates procedural context, equipment capabilities, safety protocols, and optimization targets to determine appropriate response magnitude and timing. The system may generate coordinated control signals that address multiple therapy consoles simultaneously when threshold exceedances indicate systemic adjustments are beneficial for overall procedural optimization. As mentioned, the control signals can be generated to mitigate or eliminate a deviation from one or more predetermined threshold values and/or predetermined threshold ranges.

At block 1008, “sending the control signals from the single control device to at least one therapy console of the plurality of therapy consoles to adjust an operational parameter of the at least one therapy console and mitigate a deviation from the predetermined threshold range,” the systems herein can execute automatic parameter adjustments across connected therapy equipment. The control signals can be sent via a wired and/or wireless network. The operational parameter adjustments effectuated by the control signals (e.g., upon receipt at one or more therapy consoles) may include modifications to fluid flow rates, laser energy settings, scope deflection angles, fiber positioning coordinates, pressure control parameters, temperature regulation settings, and/or visualization equipment configurations, among other possibilities. The mitigation process addresses detected deviations through targeted adjustments designed to restore operational parameters within acceptable threshold ranges while maintaining procedural effectiveness and safety standards.

At block 1010, “receiving real-time feedback information from the plurality of therapy consoles responsive to the adjustment of the operational parameter,” the system monitors the effects of implemented control actions (e.g., control signals that are transmitted) through continuous data acquisition from therapy consoles and/or therapy equipment. The real-time feedback information can include updated measurements of physiological parameters, equipment status confirmations, and/or operational effectiveness indicators, among other feedback information. In some embodiments, the real-time feedback information can include immediate response data, trending measurements over time intervals, and/or secondary (e.g., delayed) effects that may result from primary parameter adjustments across interconnected therapy console operations. For instance, the real-time feedback can be obtained subsequent to the transmission of the control signal to one or more therapy consoles to obtain updated information (real-time feedback information from the one or more therapy consoles thereby allowing the systems herein to readily evaluate the effectiveness of the control signals sent to the one more therapy consoles (e.g., in terms of mitigation of a deviation from the threshold value).

What is claimed is:

1. A method for closed loop control in a urological operating room, comprising:
receiving, at a single control device, real-time information from a plurality of therapy consoles provisioned in an operating theater, each of the plurality of therapy consoles comprising processing circuitry configured for use in performing a urological procedure;
determining whether the received information is outside a predetermined threshold range, wherein the received information comprises physiological information, captured scene information, radiological image information, procedural state information, or any combination thereof;
automatically generating control signals based on a determination that the received information is outside the predetermined threshold range;
sending the control signals from the single control device to at least one therapy console of the plurality of therapy consoles to adjust an operational parameter of the at least one therapy console and mitigate a deviation from the predetermined threshold range;
receiving real-time feedback information from the plurality of therapy consoles responsive to the adjustment of the operational parameter;
iteratively updating the control signals based on the real-time feedback information; and
sending the updated control signals to automatically adjust an operational parameter of the at least one therapy console and mitigate a remaining deviation from the predetermined threshold range.

2. The method of claim 1, wherein determining whether the received information is outside a predetermined threshold range comprises determining whether physiological information is outside a physiological threshold range, and wherein the physiological information comprises intraluminal pressure, intraluminal temperature, fluid flow rates, laser energy parameters, or any combination thereof.

3. The method of claim 1, wherein determining whether the received information is outside a predetermined threshold range comprises determining whether captured scene information is outside a visual threshold range, and wherein the captured scene information comprises turbidity, clarity, image saturation, laser aiming beam visibility, or any combination thereof.

4. The method of claim 1, wherein determining whether the received information is outside a predetermined threshold range comprises determining whether procedural state information is outside a procedural threshold range, and wherein the procedural state information comprises equipment activation status, time elapsed in procedure, treatment efficacy indicators, or any combination thereof.

5. The method of claim 1, wherein automatically adjusting an operational parameter comprises adjusting one or more operational parameters of multiple ones of the plurality of therapy consoles in a coordinated manner to optimize performance across the multiple therapy consoles.

6. The method of claim 1, wherein the plurality of therapy consoles comprises at least a fluidics unit, a laser energy console, and an endoscope, and wherein automatically adjusting an operational parameter comprises coordinating operation between at least two of the fluidics unit, laser energy console, and endoscope based on the received information.

7. The method of claim 1, further comprising:
determining additional information derived from the received information; and
adjusting the predetermined threshold range based on the additional information to provide dynamic threshold management during the urological procedure.

8. The method of claim 1, wherein automatically adjusting an operational parameter comprises adjusting fluid flow rates, laser energy parameters, scope deflection, fiber positioning, or any combination thereof.

9. The method of claim 1, further comprising:
monitoring changes in the received real-time feedback information over time;
predicting future values of the received information based on the monitored changes; and
proactively adjusting the updated control signals based on the predicted future values to prevent the received information from exceeding the predetermined threshold range.

10. The method of claim 1, further comprising:
determining stone composition and texture from the received information; and
wherein automatically adjusting an operational parameter comprises adjusting laser energy parameters based on the determined stone composition and texture to optimize stone fragmentation while maintaining the received information within the predetermined threshold range.

11. The method of claim 1, wherein the single control device is configured to automatically coordinate adjustments across multiple therapy consoles to maintain optimal procedural conditions based on the received information and real-time feedback information.

12. The method of claim 1, further comprising:
determining distance between a treatment device and target tissue from the received information; and
wherein automatically adjusting an operational parameter comprises adjusting laser energy parameters and scope positioning based on the determined distance to maintain optimal treatment conditions.

13. The method of claim 1, wherein the predetermined threshold range is dynamically adjusted during the urological procedure based on real-time analysis of the received information and real-time feedback information.

14. The method of claim 1, wherein the updated control signals comprise coordinated commands that cause synchronized automatic adjustments across multiple therapy consoles to mitigate remaining deviations from the predetermined threshold range.

15. The method of claim 1, further comprising:
generating a composite display comprising visual indications of the received information, the automatic adjustments, and the iterative closed loop control status; and
sending display control signals to cause an operating theater display to display the composite display showing real-time closed loop control operations.

16. A urological operating room closed loop control system comprising:
an operating theater;
a plurality of therapy consoles provisioned in the operating theater, each comprising processing circuitry configured for use in performing a urological procedure;
a single control device comprising:
a processor configured to:
receive real-time information from the plurality of therapy consoles;
determine whether the received information is outside a predetermined threshold range, wherein the received information comprises physiological information, captured scene information, radiological image information, procedural state information, or any combination thereof;
automatically generate control signals based on a determination that the received information is outside the predetermined threshold range;
send the control signals to at least one therapy console of the plurality of therapy consoles to adjust an operational parameter and mitigate a deviation from the predetermined threshold range;
receive real-time feedback information from the plurality of therapy consoles responsive to the adjustment;
iteratively update the control signals based on the real-time feedback information; and
send updated control signals to automatically adjust operational parameters and mitigate remaining deviations from the predetermined threshold range.

17. The urological operating room closed loop control system of claim 16, wherein the processor is further configured to automatically adjust multiple operational parameters simultaneously across different ones of the plurality of therapy consoles based on the received information and real-time feedback information to maintain optimal procedural conditions.

18. At least one machine readable storage device, comprising a plurality of instructions that in response to being executed by a processor of a single control device cause the single control device to:
receive real-time information from a plurality of therapy consoles provisioned in an operating theater, each configured for use in performing a urological procedure;
determine whether the received information is outside a predetermined threshold range, wherein the received information comprises physiological information, captured scene information, radiological image information, procedural state information, or any combination thereof;
automatically generate control signals based on a determination that the received information is outside the predetermined threshold range;
send the control signals to at least one therapy console of the plurality of therapy consoles to adjust an operational parameter and mitigate a deviation from the predetermined threshold range;
receive real-time feedback information from the plurality of therapy consoles responsive to the adjustment;
iteratively update the control signals based on the real-time feedback information; and
send updated control signals to automatically adjust operational parameters and mitigate remaining deviations from the predetermined threshold range.

19. The at least one machine readable storage device of claim 18, wherein the instructions when executed by the processor, further cause the single control device to:
monitor changes in the real-time feedback information over time;
predict future values of the received information based on the monitored changes; and
proactively adjust the updated control signals based on the predicted future values to prevent the received information from exceeding the predetermined threshold range.

20. The at least one machine readable storage device of claim 18, wherein the instructions when executed by the processor, further cause the single control device to automatically coordinate adjustments across multiple therapy consoles to maintain optimal procedural conditions based on the received information and feedback signals.
